(19) United States
(12) Patent Application Publication
(10) Pub. No.: US 2025/0223571 A1
(43) Pub. Date: Jul. 10, 2025

(54) A PROCESS FOR STORING BIOLOGICALLY ACTIVE CONSTRUCTS IN A BIODEGRADABLE MATERIAL

(71) Applicant: BIONEEDLE DRUG DELIVERY B.V., LEIDEN (NL)

(72) Inventor: Gijsbert VAN DE WIJDEVEN, LEIDEN (NL)

(21) Appl. No.: 18/852,352

(22) PCT Filed: Mar. 30, 2023
(86) PCT No.: PCT/NL2023/050165

§ 371 Date: Sep. 27, 2024

(30) Foreign Application Priority Data
Mar. 30, 2022 (NL) 2031451

(51) Int. Cl. C12N 7/00

(52) U.S. Cl. CPC C12N 7/00; C12N 2710/10051; C12N 2760/18051; C12N 2770/36051

(57) ABSTRACT

A process is provided for storing a lipid containing composition in a biodegradable material, the method comprising the steps: a) providing a biodegradable material, wherein the biodegradable material comprises or consists essentially of a processed starch; b) providing the lipid containing composition, wherein the lipid containing composition comprises lipid nanoparticles, preferably providing a liquid formulation comprising the lipid containing composition and a liquid carrier; c) absorbing said a lipid containing composition into the biodegradable material; and d) storing the lipid containing composition in the biodegradable material at a storage temperature from −80° C. to 80° C. for a period of at least one day. The invention further provides a product obtainable by the process of any one of the preceding claims, the product comprising said lipid containing composition comprising lipid nanoparticles, which are accommodated inside said biodegradable material.

### FIELD OF THE INVENTION

The present invention relates to storing biologically active constructs in a biodegradable material. The invention further relates to a product comprising said biodegradable material and said biologically active constructs. The invention further relates to uses of said product.

### BACKGROUND ART

This invention relates to the stabilization of biologically active constructs as used mainly but not exclusively for vaccines. A given vaccine is a substance that, when brought into contact with the immune system of a healthy body, induces a reaction by the immune system directed against a specific pathogen. The immune system reacts by building proteinaceous antibodies [humoral immunity] and/or by sensitizing immune cells that attack the pathogen [cellular immunity]. Both humoral and cellular reactions by the immune system are dependent on the three dimensional [tertiary] molecular structure of specific proteins of the pathogen. These specific protein molecules have specific regions, called epitopes, that are being recognised by the immune system and against which the antibodies are directed.

For an overview of vaccines and vaccine platforms reference is made to the article “Vaccine instability in the cold chain: Mechanisms, analysis and formulation strategies” by Ozan S. cs. This is an overview of around 40 much used proteinaceous, viral and bacterial vaccines and the characteristics of each; the overview indicates that all vaccines are either liquid or lyophilized and they must be kept either frozen or at 2-8° C.; limited stability is due to heat and cold sensitivity.

Since the COVID19 pandemic a new vaccine platform has been introduced: mRNA-LNP's [messenger ribonucleic acid-lipid nanoparticles]: these are nano particles [size in the magnitude of 100 nm] with a membrane that resembles cell walls, containing fatty substances [lipids] and messenger RNA; after injection the mRNA-LNP's are being endocytosed [taken up by cells], delivering the mRNA's in the cytosol to the ribosomes to produce the proteins with the epitopes, which then are presented at the cell surface to stimulate the immune system; examples: mRNA-LNP vaccines against COVID19.

Lipid containing compositions which comprise lipid nanoparticles are developed in general and known for enhancing or supporting the effective delivery of nucleic acids to appropriate sites within a cell or organism in order to realize the potential of using nucleic acids, such as is described in WO2017004143A1.

To explain the reach of the current invention regarding the stabilisation of vaccines, it is useful to put these vaccine platforms into three distinct groups:
1. proteinaceous vaccines [toxoids, subunit proteins, WIV, Split, recombinant proteins] as non-living biologically active constructs;
2. infectious [“live”] viral vaccines [LAV, recombinant adenovirus vector] as living biologically active constructs;
3. mRNA-LNP's as artificial biologically active constructs; part of this group are also liposomal vaccines, which also have membrane structures;
The common feature of all three groups is their biological nature, built of complex molecules [with tertiary structures] and of complexes of complex molecules [which are called quaternary structures], with either proteins with their epitopes or carrying the genetic information to produce such proteins; therefore the term “biologically active construct” is used here to refer to all three groups.

A common problem for storing and transporting formulations containing biologically active constructs is their limited thermostability.

As to the artificial biologically active constructs mRNA-LNP's, these are vaccines that are unstable for several reasons, as explained in e.g. the recent scientific article “mRNA-lipid nanoparticle COVID-19 vaccines: Structure and stability” by Linde Schoenmaker et al [2021]; this article describes also the structure of the mRNA-LNP's; mRNA-LNP's do not contain proteins, so their thermosensitivity has different causes. The mRNA-strands are extremely sensitive to enzymatic hydrolysis as well as to chemical hydrolysis in the presence of Bronsted salts; hydrolysis leads to breakage of the RNA chain, with as a result that it cannot be translated into proteins. Secondly the single stranded mRNA-molecules “melt” and unfold at certain temperatures, losing their secondary structure [as in proteins] with increased risk of hydrolysis; there are different thermal unfolding temperatures of different RNA's: e.g. ribosomal RNA is stable until 70° C., whereas m-RNA loses its secondary structure at 32° C., see “Genome-wide Measurement of RNA Folding Energies” by Yue Wan et al [2012]. Thirdly, by their nature the LNP's tend to aggregate or to fuse within minutes. In the fourth place at lowering pH the cationic ionizable lipids show hexagonal transformation, whereby the LNP is disrupted and the mRNA released before it can be endocytosed by cells, see e.g. “Lipid Nanoparticle Systems for Enabling Gene Therapies” by Pieter R. Cullis et al [2017]. A fifth reason is that lipids can be oxidated, which is an autocatalyzing process with exponential increase after days to weeks. Sixthly, the LNP's are sensitive to physical stress such as shear and shaking. In the 7th place desintegration and loss can occur of the PEG-ylated lipids. Finally domain formation can occur in the presence of free lipids, changing the capacity of the LNP's to be endocytosed, see e.g. “Polyunsaturated fatty acid-cholesterol interactions: Domain formation in membranes” by Stephen R. Wassall et al [2008]. Although mRNA can be stabilized by freeze-drying, it has been shown that LNP's cannot be freeze-dried [reference is made to L. Schoenmaker c.s.].

All these 8 reasons together have the consequence that all mRNA-LNP vaccines must be kept in the range of temperatures from minus 20° C. [Moderna] to minus 90° C. [BioNTech/Pfizer] during transport from manufacturer, during storage at the national distribution sites and during distribution to and storage at the Health Centres until a couple of hours prior to injection. It is concluded that there is a need for a new technology that allows to stabilize mRNA-LNP vaccines; it is suggested that thermostability could be enhanced by either smart sequencing of the mRNA replacing ribonucleic acids moieties for increasing the unfolding temperature] and/or by improved freeze drying. Overall, it can be stated that the stability of the mRNA-LNP vaccine platform is of more difficult order than all other vaccine platforms.

Overall, all vaccines are thermosensitive and need to be kept at either 2° C.-8° C. or at minus 20° C. to minus 90° C. depending on the vaccine platform.

The need for refrigeration or freezing during transport, storage and distribution [the “cold chain”] of all types of vaccines is a major drawback, because in practice it appears very difficult and often impossible to continuously maintain the cold chain from the vaccine manufacturer until the moment of delivery to the patients, especially in Low and Middle Income Countries [LMIC's].

Therefore, there is a need for an innovative technology to improve vaccine thermostability in order to equally reach all people. Although much effort is put in the development of thermostability of specific vaccines, there is no vaccine thermostability technology established as yet that can stabilize all types of vaccines.

It therefore would be advantageous if a technology could be developed that can be used to stabilize all three types of vaccines [live vaccines, mRNA-LNP's and proteinaceous vaccines] and simultaneously bring the vaccine into a presentation that can directly be delivered to patients, so that vaccine manufacturers do not have to develop such stabilization technology for each vaccine separately.

There remains a need for improved means and methods for storing and transporting biologically active constructs, in particular lipid containing compositions which include lipid nanoparticles, e.g. for the delivery of RNA and DNA. Especially for storing and stabilizing lipid containing compositions at higher temperatures, such is higher than minus 20° C., there remains a need for improved means and methods for storing and transporting biologically active constructs.

US 2016/0206615 describes pharmaceutical formulations of inhibitors for poly (ADP-ribose) polymerase (PARP) enzyme.

US 2020/129615 relates to herpes simplex virus (HSV) ribonucleic acid (RNA) vaccines, as well as vaccines and compositions comprising the vaccines.

US 2014/287043 describes methods and compositions for stabilization of active agents.

WO 2008/105663 relates to a kinetic implant comprising (a) biodegradable material comprising opened starch, destructurised starch or a mixture of opened starch and destructurised starch, (b) a biologically or pharmaceutically active substance, and (c) a stabilizing component stabilising the biologically or pharmaceutically active substance.

Muramatsu et al. (Molecular Therapy, 2022, vol. 30, no. 5, pgs. 1941-1951) describe a method for stabilizing a lipid nanoparticle-formulated, nucleoside-modified mRNA vaccine.

### SUMMARY OF THE INVENTION

The present invention aims to provide a process for storing biologically active constructs, wherein the stability, in particular the thermostability, of the biologically active constructs is improved.

In preferred embodiments, the invention aims to provide a process for storing a lipid containing composition, wherein the stability, in particular the thermostability, of the lipid containing composition is improved.

In preferred embodiments, the invention aims to provide a product containing a lipid containing composition, wherein the product is easily transportable.

According to the present invention is provided a process for storing a lipid containing composition in a biodegradable material, the method comprising the steps:
a) providing a biodegradable material, wherein the biodegradable material comprises or consists essentially of a processed starch;
b) providing the lipid containing composition, wherein the lipid containing composition comprises lipid nanoparticles, preferably providing a liquid formulation comprising the lipid containing composition and a liquid carrier;
c) absorbing said a lipid containing composition into the biodegradable material; and
d) storing the lipid containing composition in the biodegradable material at a storage temperature from −80° C. to 80° C. for a period of at least 1 day.

In another aspect, the invention provides a product obtainable by the process of any one of the preceding claims, the product comprising said lipid containing composition comprising lipid nanoparticles, which are accommodated inside said biodegradable material.

In another aspect, the invention provides a product obtained by the process of any one of the preceding claims, the product comprising said lipid containing composition comprising lipid nanoparticles, which are accommodated inside said biodegradable material.

In another aspect, the invention provides a use of the product according to the invention, wherein the use comprises releasing the lipid containing composition comprising lipid nanoparticles from the biodegradable material after said storing step by reconstituting the biodegradable material in a water containing reconstitution liquid.

In another aspect, the invention provides a process of using the product according to the invention, wherein the process comprises releasing the lipid containing composition comprising lipid nanoparticles from the biodegradable material after said storing step by reconstituting the biodegradable material in a water containing reconstitution liquid.

Surprisingly it was discovered that a biodegradable material, wherein the biodegradable material comprises or consists essentially of a processed starch, can be used to stabilize the biologically active constructs, in particular lipid containing composition, based on the condition that the biodegradable material absorbs the biologically active constructs.

Data were generated showing that the biodegradable material as such can improve the thermo-, heat-, freeze- and storage stabilities of mRNA-LNP's, when the mRNA-LNP's are contained inside the biodegradable material, as shown in the examples given further below.

Once the biodegradable material has been filled with the liquid formulation containing the lipid containing composition, the lipid containing composition is stabilized, and the product (i.e. biodegradable article containing said lipid containing composition), which is obtained by the process, can be transported, stored and distributed outside the cold chain. When a person is to be vaccinated with the mRNA-LNP's, the stabilized vaccine containing biodegradable article can either be kinetically applied through skin [where the biodegradable material article will absorb interstitial fluids=fluids between the cells] or [prior to injection] be put into excess of water, allowing the biodegradable article to dissolve the amylose, and allowing the amylopectin to form a gel, where simultaneously the vaccine will be reconstituted and released from the amylopectin gel.

A biodegradable material, usable for the invention, which comprises processed starch is known from PCT/NL2008/050120. The biodegradable material is an excellent starting material for manufacturing biodegradable shaped articles, for example by injection moulding, wherein said biodegradable shaped articles are suitable for delivery of a biologically or pharmaceutically active component in or to a vertebrate, e.g. a mammal. The biodegradable material has a low cytotoxicity. The biodegradable shaped articles are in particular suitable for parenteral, oral, transdermal, subcutaneous and hypodermic applications.

In a preferred exemplary embodiment, the absorbing step of the lipid containing composition is based on absorbing a liquid formulation comprising the lipid containing composition and a liquid carrier, preferably the liquid carrier comprising water. Said liquid formulation is an aqueous formulation. In embodiments, the water concentration of the aqueous formulation is in the range of 1-99 wt. %, preferably 10-90 wt. %, more preferably 10-50 wt. %.

Said water may be H₂O and/or may be D₂O.

In an exemplary embodiment, the absorbing step has a duration of 0.1 second-24 hours, preferably 1 second to 60 minutes, more preferably at least 5 seconds, in particular at least 10 seconds, more preferably at most 30 minutes. The absorbing step may have a duration of at least 30 seconds, in particular 1 minute, more in particular at least 3 minutes. The absorbing step may have a duration of at most 20 minutes, in particular at most 15 minutes, more in particular at most minutes, even more in particular at most 5 minutes.

Preferably, the absorption step comprises absorbing at least 1.0 vol. %, in particular at least 5.0 vol. %, more in particular at least 10 vol. % of the liquid formulation, more preferably absorbing at least 50 vol. % of the liquid formulation, more in particular 80 vol. % of the liquid formulation.

In an exemplary embodiment, the process comprises stabilising the lipid containing composition by accommodating the a lipid containing composition inside the biodegradable material, preferably thermostabilising the lipid containing composition, preferably stabilising by accommodating at least a part of the lipid containing composition between amylopectin layers being present in the biodegradable material.

In an exemplary embodiment, the biodegradable material has a water content of less than 70 wt. % directly after the absorbing step, based on the total weight of the biodegradable material, preferably wherein the biodegradable material has a water content of less than 60 wt. % directly after the absorbing step, preferably less than 50 wt. %.

Without being bound to theory, the preferred amount of water content of the biodegradable material directly after the absorbing step of the liquid formulation may be due to a better distribution of the absorbed lipid containing composition, in particular lipid nanoparticles, into the biodegradable material.

Preferably, the biodegradable material is provided in a substantially dry solid state, with a water content of less than 10 wt. % at the start of the absorption step, more preferably less than 5 wt. %, more preferably less than 3 wt. %, based on the total weight of the biodegradable material. This supports for being able to absorb the solution or suspension of the biologically active constructs and retain the water content of less than 70 wt. % directly after the absorbing step.

In an exemplary embodiment, the process further comprises cooling the biodegradable material after the absorbing step, preferably by using snap-freezing, to a cooling temperature of −70° C. to −30° C. The cooling step is preferably carried out before the storing step.

In a preferred exemplary embodiment, the snap-freezing step to said cooling temperature is performed within 0.1 seconds-30 seconds, preferably within 10 seconds, more preferably within 5 seconds.

The snap-freezing step is selected for rapidly cooling the biodegradable material such that substantially no ice crystals are formed inside the biodegradable material.

Surprisingly we have found that the lipid nanoparticles can be cooled to a cooling temperature of −70° C. to −30° C., e.g. using snap-freezing, without disturbing the lipid nanoparticles, when the lipid nanoparticles are contained inside the biodegradable material.

In an exemplary embodiment, the process further comprises drying the biodegradable material after the absorbing step, preferably wherein the drying step is or comprises freeze-drying the biodegradable material, optionally to a water content of less than 10 wt. %, preferably less than 5 wt. %, preferably less than 3 wt. %, preferably less than 1 wt. %.

In an exemplary embodiment, the storage temperature is −80° C. to 80° C., preferably −20° C. to 60° C., more preferably 20° C. to 60° C., in particular 30° C. to 50° C., or more preferably 0° C. to 20° C., in particular 2° C. to 10° C.

In an exemplary embodiment, the storing is for a period of at least two days till at most 5 years, preferably for at least three days, more preferably for at least one month, in particular for at least two months or for at least 6 months or for at least one year, and/or
preferably for at most five years, more preferably for at most one year, in particular for at most 6 months or for at most one month.

In a preferred exemplary embodiment, the storage step comprises transporting the biodegradable material including the lipid containing composition, preferably at conditions of time and temperature suitable for easily transporting and storing the biodegradable material. For example, transporting and storing at a temperature above e.g. 0° C. has the advantage that simple facilities can be used, without cooling the biodegradable material including the lipid containing composition to a lower temperature. The biodegradable material provides a thermostabilised lipid containing composition, which can be easily transported at relatively high temperatures.

In an exemplary embodiment, the biodegradable material is a processed starch comprising amylopectin layers, which amylopectin layers preferably have an interlayer distance in the range of 10 nm-300 nm and/or which amylopectin layers preferably have a thickness in the range of 100-800 nm, preferably 100-500 nm.

In an exemplary embodiment, the biodegradable material is a pregelatinized starch composition and/or a thermoplastic starch composition, preferably comprising a layered phase comprising amylopectin layers and a homogenous amylose phase, wherein more preferably the layered phase is at least 10 to 90 wt. % based on the total weight of the biodegradable material.

Said biodegradable material easily absorbs water at ambient temperatures, for example temperatures between 0 and 40 degrees Celsius, and forms a gel phase.

Said layered phase is a discrete phase, which also is referred to as a block which is dispersed within the homogenous amylose phase. A plurality of layered phases or blocks are typically distributed throughout the homogenous amylose phase.

The homogenous amylose phase contains amorphous amylose in a glassy state and contains substantially no amylopectin layers. The homogenous amylose phase may additionally contain amylopectin components, which are not arranged in layers, and may contain smaller carbohydrates in a glassy state.

The total of layered phase and homogenous amylose phase is referred to as amylomatrix.

The processed amylopectin of the layered phase according to the present invention has preferably a weight average molecular weight of about 20.000.000 to about 100.000.000 as determined by MALLS (Multi Angle Laser Light Scattering) on samples that were obtained after DMSO solubilisation and precipitation in alcohol.

The molecular weight distribution Mw/Mn of the processed amylose is preferably in the range of about 2 to about 3. The weight average molecular weight of the processed amylose is preferably in the range of about 500.000 to about 2.000.000.

In an exemplary embodiment, the biodegradable material has a bulk density of 1.0 to 1.5 kg/dm3.

In an exemplary embodiment, the liquid formulation, which comprises the lipid nanoparticles, is selected from an emulsion of the lipid nanoparticles in the liquid carrier or a suspension of the lipid nanoparticles in the liquid carrier.

In an exemplary embodiment, the lipid nanoparticles have a particle size in the range of 5 nm-300 nm, preferably 10 nm-200 nm as measured by Dynamic Light Scattering (e.g. using a Zetasizer Pro Red Light Scattering System, Advance Series (Malvern Analytics)). For example, the particle size may be an average particle size in the range of 50 to 200 nm, in particular in the range of 60 to 150 nm, more in particular in the range of 90 to 120 nm.

It has been found that the process according to the invention provides the advantage that the particle size of the nanoparticles is substantially unaffected by the storage conditions of temperature and period. Thus, the particle size of the nanoparticles is substantially the same for the initial particle size before and the final particle size after the storing and reconstitution steps.

In an exemplary embodiment, the lipid containing composition further comprises a pharmaceutically active agent. Preferably the pharmaceutically active agent comprises or is a Ribonucleic acid {RNA] or desoxyribonucleic acid [DNA].

In an exemplary embodiment, the lipid nanoparticles comprise one or more excipients selected from neutral lipids, cationic ionisable lipids, steroids [such as cholesterol], polymer conjugated lipids and conjugated lipids having a hydrophilic moiety.

In embodiments, the polymer conjugated lipids may have a polymer conjugated to the lipid, wherein the polymer is or comprises a hydrophilic moiety providing hydrophilic properties to the conjugated lipids. For example, a pegylated lipid is such a conjugated lipids having a hydrophilic moiety.

In an exemplary embodiment, the lipid nanoparticles comprise one or more neutral lipids selected from 1,2-distearoyl-sn-glycero-3-phosphocholine (DSPC), 1,2-dipalmitoyl-sn-glycero-3-phosphocholine (DPPC), 1,2-dimyristoyl-sn-glycero-3-phosphocholine (DMPC), 1,2-dioleoyl-sn-glycero-3-phosphocholine (DOPC), 1-palmitoyl-2-oleoyl-sn-glycero-3-phosphocholine (POPC), 1,2-dioleoyl-sn-glycero-3-phosphoethanolamine (DOPE) and sphingomyelin (SM).

In an exemplary embodiment, the polymer conjugated lipid is a pegylated lipid, preferably wherein the pegylated lipid is pegylated diacylglycerol (PEG-DAG), pegylated phosphatidylethanolamine (PEG-PE), polyethylene glycol succinate diactylglycerol (PEG-S-DAG), pegylated ceramide (PEG-cer) or a polyethylene glycol (PEG) dialkyloxypropylcarbamate.

In an exemplary embodiment, the pharmaceutically active agent comprises a nucleic acid, preferably wherein the nucleic acid is selected from DNA and RNA, preferably any one of silencing RNA, antisense RNA and messenger RNA. Said messenger RNA may be self-amplifying mRNA.

In an exemplary embodiment, the biodegradable material is provided as a shaped article, preferably a moulded or extruded article.

In an exemplary embodiment, the shaped article is selected from a hollow cylinder, a tablet, a hollow capsule, a kinetic implant and a rod shaped article.

In an exemplary embodiment, the absorbing step comprises applying the liquid formulation comprising the lipid containing composition and the liquid carrier onto or into the shaped article of the biodegradable material.

In an exemplary embodiment of the process and/or of the product, at least 50% of the lipid nanoparticles have a particle size in the range of 5 nm-300 nm, preferably 10 nm-200 nm, after the storage step, preferably at least 80%, more preferably at least 90%, wherein the percentage is a number %.

In an exemplary embodiment of the process and/or of the product, the lipid nanoparticles are at least partly present in the layered phase of the biodegradable material comprising amylopectin layers, wherein the percentage is a number %.

Preferably at least 50% of the lipid nanoparticles, more preferably at least 80%, are present in the layered phase of the biodegradable material comprising amylopectin layers.

In particular, at least 30% of the lipid nanoparticles are accommodated between amylopectin layers of the layered phase, wherein the percentage is a number %.

Preferably at least 50% of the lipid nanoparticles, more preferably at least 80%, are accommodated between amylopectin layers of the layered phase.

In an exemplary embodiment of the process and/or of the product, the lipid nanoparticles contain a pharmaceutically active agent, wherein after the storage step at least 10% of the lipid nanoparticles contain said pharmaceutically active agent, preferably at least 50%, more preferably at least 80%, wherein the percentage is a number %.

In an exemplary embodiment of the process and/or of the product, the product is a shaped article, preferably a shaped article is selected from a hollow cylinder, a tablet, a hollow capsule, a kinetic implant, and a rod shaped article.

In an exemplary embodiment of the process and/or of the product, the product is a kinetic implant for implanting into a body.

In an exemplary embodiment of the process and/or of the product, the product is a tablet or a capsule for oral administration.

In another part of the invention, there is provided the following aspects and embodiments:

1. A process for storing biologically active constructs in a biodegradable material, the method comprising the steps:
a) providing a biodegradable material, wherein the biodegradable material comprises or consists essentially of a processed starch;
b) providing biologically active constructs, preferably providing a liquid formulation comprising the biologically active constructs and a liquid carrier;
c) absorbing said biologically active constructs into the biodegradable material; and
d) storing the biologically active constructs in the biodegradable material at a storage temperature from −80° C. to 80° C. for a period of at least 1 day.

In a preferred embodiment of the invention, the liquid formulation, including its water content, and the relative amounts (in weight) of the liquid formulation and the biodegradable material, are selected such that the biodegradable material has a water content of less than 70 wt. % directly after the absorbing step, based on the total weight of the biodegradable material.

2. process for storing biologically active constructs in a biodegradable material, wherein the absorbing step of the biologically active constructs is based on absorbing a liquid formulation comprising the biologically active constructs and a liquid carrier, preferably the liquid carrier comprising water.

3. process for storing biologically active constructs in a biodegradable material, wherein the absorbing step has a duration of 0.1 second-24 hours, preferably 1 second to 60 minutes, more preferably at least 5 seconds, in particular at least 10 seconds, more preferably at most 30 minutes.

4. process for storing biologically active constructs in a biodegradable material, wherein the process comprises stabilising the biologically active constructs by accommodating the biologically active constructs inside the biodegradable material, preferably thermostabilising the biologically active constructs, preferably stabilising by accommodating at least a part of the biologically active constructs between amylopectin layers being present in the biodegradable material.

5. process for storing biologically active constructs in a biodegradable material, wherein the biodegradable material has a water content of less than 70 wt. % directly after the absorbing step, based on the total weight of the biodegradable material, preferably wherein the biodegradable material has a water content of less than 60 wt. % directly after the absorbing step, preferably less than 50 wt. %.

6. process for storing biologically active constructs in a biodegradable material, wherein the process further comprises cooling the biodegradable material after the absorbing step, preferably by using snap-freezing, to a cooling temperature of −70° C. to −30° C.

7. process for storing biologically active constructs in a biodegradable material, wherein the snap-freezing step to said cooling temperature is performed within a period of 0.1 seconds-30 seconds, preferably within 10 seconds, more preferably within 5 seconds.

8. process for storing biologically active constructs in a biodegradable material, wherein the process further comprises drying the biodegradable material after the absorbing step, preferably wherein the drying step is or comprises freeze-drying the biodegradable material, optionally to a water content of less than 10 wt. %, preferably less than 5 wt. %, preferably less than 3 wt. %, preferably less than 1 wt. %.

9. process for storing biologically active constructs in a biodegradable material, wherein the storage temperature is −80° C. to 80° C., preferably −20° C. to 60° C., more preferably 20° C. to 60° C., in particular 30° C. to 50° C., or more preferably 0° C. to 20° C., in particular 2° C. to 10° C.

10. process for storing biologically active constructs in a biodegradable material, wherein the storing is for a period of at least two days till at most 5 years, preferably for at least three days, more preferably for at least one month, in particular for at least two months or for at least 6 months or for at least one year, and/or preferably for at most five years, more preferably for at most one year, in particular for at most 6 months or for at most one month.

11. process for storing biologically active constructs in a biodegradable material, wherein the biodegradable material is a processed starch comprising amylopectin layers, which amylopectin layers preferably have an interlayer distance in the range of 10 nm-300 nm and/or which amylopectin layers preferably have a thickness in the range of 100-800 nm, preferably 100-500 nm.

12. process for storing biologically active constructs in a biodegradable material, wherein the biodegradable material is a pregelatinized starch composition and/or a thermoplastic starch composition, preferably comprising a layered phase comprising amylopectin layers and a homogenous amylose phase, wherein more preferably the layered phase is at least 10 to 90 wt. % based on the total weight of the biodegradable material.

13. process for storing biologically active constructs in a biodegradable material, wherein the biodegradable material has a bulk density of 1.0 to 1.5 kg/dm3.

14. process for storing biologically active constructs in a biodegradable material, wherein the liquid formulation, which comprises the biologically active constructs, is selected from a solution of the biologically active constructs in the liquid carrier, an emulsion of the biologically active constructs in the liquid carrier or a suspension of the biologically active constructs in the liquid carrier.

15. process for storing biologically active constructs in a biodegradable material, wherein the biologically active constructs have a particle size in the range of 5 nm-500 nm, preferably 10 nm-300 nm.

16. process for storing biologically active constructs in a biodegradable material, wherein the biologically active constructs further comprise a pharmaceutically active agent.

17. process for storing biologically active constructs in a biodegradable material, wherein the pharmaceutically active agent comprises any one of a proteinaceous construct, such as a proteinaceous vaccine, including toxoids, subunit proteins, WIV, Split, recombinant proteins, infectious viral vaccines, including LAV, and recombinant adenovirus vector.

18. process for storing biologically active constructs in a biodegradable material, wherein the vaccine is any one selected of:
a. LAV=Live Attenuated Viral vaccines [pathogenic live virus that has been attenuated, id est that has been made apathogenic, but still can replicate in the body and present the proteins with the epitopes to the immune system]; for example Measles;
b. WIV=Whole Inactivated Viral vaccines [with the proteins with the epitopes at their surface]; example: Influenza A;
c. Split Virus Vaccines [=broken virus particles, with the proteins with the epitopes]; e.g. Influenza A;
d. Subunit Proteins Vaccines [=purified proteins from the inactivated virus and with the epitopes]; e.g. Influenza A;
e. VLP's [Virus Like Particles, or Virosomes=artificial constructs of auto-assembling viral envelope incorporating the epitopic protein at their surface]; e.g. Influenza A;
f. Recombinant Protein Vaccines [produced by cells or microorganisms that are genetically modified by the insertion of the genetic code coding for the protein with the epitope]; e.g. Hepatitis B;
g. Toxoids=poisonous proteins with epitopes; example: Tetanus toxoid; Typhoid;
h. live apathogenic Adenovirus Vectors [non pathogenic dsDNA viruses that are genetically modified with the code for the protein with the epitope; after injection into a healthy body, these virus particles invade cells and stimulate these cells to produce the proteins with the epitopes which then are being presented to the immune system]; example: adenoviral vaccines against COVID19;
i. mRNA-LNP's [messenger ribonucleic acid-lipid nano particles]: these are nano particles with a membrane containing fatty substances [lipids] and messenger RNA; after injection the mRNA-LNP's are being endocytosed [taken up by cells], delivering the mRNA's in the cytosol to the ribosomes to produce the proteins with the epitopes, which then are presented at the cell surface to stimulate the immune system; examples: the mRNA-LNP vaccine against COVID19.

19. process for storing biologically active constructs in a biodegradable material, wherein the biodegradable material is provided as a shaped article, preferably a moulded or extruded article.

20. process for storing biologically active constructs in a biodegradable material, wherein the shaped article is selected from a hollow cylinder, a tablet, a hollow capsule, a kinetic implant and a rod shaped article.

21. process for storing biologically active constructs in a biodegradable material, wherein the absorbing step comprises applying the liquid formulation comprising the biologically active constructs and the liquid carrier onto or into the shaped article of the biodegradable material.

22. A product obtainable by the process for storing biologically active constructs in a biodegradable material, the product comprising said biologically active constructs, which are accommodated inside said biodegradable material.

23. The product, wherein at least 50% of the biologically active constructs have a particle size in the range of 5 nm-300 nm, preferably 10 nm-200 nm, after the storage step, preferably at least 80%, more preferably at least 90%, wherein the percentage is a number %.

24. The product, wherein the biologically active constructs are at least partly present in the layered phase of the biodegradable material comprising amylopectin layers, in particular at least 30% of the biologically active constructs are accommodated between amylopectin layers of the layered phase.

25. The product, wherein the biologically active constructs contain a pharmaceutically active agent, wherein after the storage step at least 10% of the biologically active constructs contain said pharmaceutically active agent, preferably at least 50%, more preferably at least 80%, wherein the percentage is a number %.

26. The product, wherein the product is a shaped article, preferably a shaped article is selected from a hollow cylinder, a tablet, a hollow capsule, a kinetic implant, and a rod shaped article.

27. The product, wherein the product is a kinetic Implant for implanting into a body.

28. The product, wherein the product is a tablet or a capsule for oral administration.

29. Use of the product, wherein the use comprises releasing the biologically active constructs from the biodegradable material after said storing step by reconstituting the biodegradable material in a water containing reconstitution liquid.

30. Use of the product, wherein the reconstitution liquid further comprises an enzyme for hydrolysing starch, such as amylase, e.g. serum amylase.

31. Use of the product, wherein the reconstitution step is carried out in a syringe.

### Definitions

### Biodegradable Material

The biodegradable material according to the invention comprises starch of a particular physical state, i.e. a processed starch. The biodegradable material is an excellent starting material for manufacturing biodegradable shaped articles, for example by injection moulding.

The biodegradability relates to a very fast degradation; fast degradation is a desirable effect for the present invention.

Biodegradable materials based on native starch, (chemically) modified starch and similar substances are commonly known in the art.

The term “native starch” is to be understood as a native starch material that is obtained from seeds and cereals, e.g. corn, waxy corn, high amylose corn, oats, rye, maize, wheat and rice, or roots, e.g. potato, sweet potato and tapioca. Preferably, the starch material is potato starch, maize starch or corn starch, most preferably potato starch.

It is further well known that the main components of native starch material are amylose and amylopectin, the molecular weights thereof being dependent from the origin of the starch (cf for example Kirk-Othmer, Encyclopedia of Chemical Technology, Vol. 22, 699-719, 1997).

The prior art, in particular in EP A 774.975, incorporated by reference herein, discloses “destructurised starch” and “substantially destructurised starch” which implies that essentially all starch particles are destructurised, i.e. all starch granules are disrupted, and within the disrupted starch granules, the starch molecules are dispersed. In this document, the materials “destructurised starch” and “substantially destructurised starch” are indicated by the generic term “destructurised starch” for convenience.

Additionally, a destructurised starch is understood to be substantially without any structured or layered amylopectin.

### Processed Starch

Processed starch is a starch, preferably a native starch, which has been processed by a combination of temperature and shear to at least partly change, i.e. breakdown, the original structure of the native starch. The processed starch has at least a partly disruption of the starch granules.

In processed starch according to the invention, the amylopectin layers stay at least partly intact, although the hydrogen bonds that were originally present in between the amylopectin layers may be broken. The processed starch contains typically a high number of layered phases or layered domains, distributed throughout a matrix of homogeneous amylose phase, which mainly contains amylose.

The processed starch of the invention is also referred to throughout the description as “amylomatrix”.

By the term “processed” as used in this document regarding the amylose and amylopectin components, it is intended to indicate that these components are different from the amylose and amylopectin as they occur in the native starch, i.e. that during processing some degradation or modification may have occurred.

### Pregelatinized Starch Composition

Pregelatinized starch is generally known as starch which has been heat treated, e.g. by cooking, and then dried in a starch factory, e.g. on a drum dryer, or in an extruder, making the starch cold-water-soluble to form a gel.

Spray dryers are used to obtain dry starch particles and low viscous pregelatinized starch powder. Pregelatinized starch compositions are further described in “Starch Chemistry and Technology; third edition” by James BeMiller and Roy Whistler.

### Thermoplastic Starch Composition

Plastics are a wide range of materials that use polymers as a main ingredient. Their plasticity makes it possible for plastics to be moulded, extruded or pressed into solid objects of various shapes. Pure starch-based bioplastic is brittle. Plasticizers such as glycerol, glycol, and sorbitol can also be added so that the starch can also be processed thermo-plastically. The characteristics of the resulting bioplastic (also called “thermoplastic starch”) can be tailored to specific needs by adjusting the amounts of these additives. Conventional polymer processing techniques can be used to process starch into bioplastic, such as extrusion, injection moulding, compression moulding and solution casting.” Further general description of thermoplastic starches can be found in the handbook: “Starch Chemistry and Technology”.

### Biologically Active Constructs

The common feature of biologically active constructs is their biological nature, built of complex molecules [with tertiary structures] and of complexes of complex molecules [which are called quaternary structures], with either proteins with their epitopes or carrying the genetic information to produce such proteins; therefore the term “biologically active construct” is used here to refer to all three groups of the following groups of vaccines:
1. proteinaceous vaccines [toxoids, subunit proteins, WIV, Split, recombinant proteins] as non-living biologically active constructs;
2. infectious [“live”] viral vaccines [LAV, recombinant adenovirus vector] as living biologically active constructs;
3. mRNA-LNP's as artificial biologically active constructs; part of this group are also liposomal vaccines, which also have membrane structures.

However, biologically active constructs are in principle not limited to vaccines. In principle biologically active constructs can be any pharmaceutically active constructs, which are based on pharmaceutically active agents, e.g. proteinaceous substances, viral substances and artificial biologically active constructs, such as LNP-based compositions.

### Vaccines

For an overview of vaccines and vaccine platforms reference is made to the article “Vaccine instability in the cold chain: Mechanisms, analysis and formulation strategies” by Ozan S. cs. The following vaccine platforms can be defined:
Live and inactivated bacterial vaccines [e,g. BCG] carrying proteins with their epitopes;
LAV=Live Attenuated Viral vaccines [pathogenic live virus that has been attenuated, id est that has been made apathogenic, but still can replicate in the body and present the proteins with the epitopes to the immune system]; example: Measles
WIV=Whole Inactivated Viral vaccines [with the proteins with the epitopes at their surface]; example: Influenza A; [also inactivated bacteria]
Split Virus Vaccines [=broken virus particles, with the proteins with the epitopes]; e.g. Influenza A;
Subunit Proteins Vaccines [=purified proteins from the inactivated virus and with the epitopes]; e.g. Influenza A;
VLP's [Virus Like Particles, or Virosomes=artificial constructs of auto-assembling viral envelope incorporating the epitopic protein at their surface]; e.g. Influenza A;
Recombinant Protein Vaccines [produced by cells or microorganisms that are genetically modified by the insertion of the genetic code coding for the protein with the epitope]; e.g. Hepatitis B;
Toxoids=poisonous proteins with epitopes; example: Tetanus toxoid; Typhoid;
Live apathogenic Adenovirus Vectors [non pathogenic dsDNA viruses that are genetically modified with the code for the protein with the epitope; after injection into a healthy body, these virus particles invade cells and stimulate these cells to produce the proteins with the epitopes which then are being presented to the immune system]; example: adenoviral vaccines against COVID-19;
mRNA-LNP's [messenger ribonucleic acid-lipid nano particles]: these are nano particles [100 nm] with a membrane containing fatty substances [lipids] and messenger RNA; after injection the mRNA-LNP's are being endocytosed [taken up by cells], delivering the mRNA's in the cytosol to the ribosomes to produce the proteins with the epitopes, which then are presented at the cell surface to stimulate the immune system; examples: the mRNA-LNP vaccine against COVID-19. For an overview of vaccines which are developed towards COVID-19, we refer to the review article “Reasons for success and lessons learnt from nanoscale vaccines against COVID-19” [August 2021] from Thomas Kisby, Açelya Yilmazer and Kostas Kostarelos, wherein vaccines against COVID-19 are described. The review article describes reasons behind the success of such advanced nanoscale vaccine technologies compared with clinically established conventional vaccines, and the lessons to be learnt from this potentially transformative development in the adoption and acceptance of nanotechnology for medicine. The vaccines described in this article are incorporated by reference.

### Lipid Nanoparticles

These are nanoparticles with a membrane containing fatty substances [lipids]. As the skilled person will understand, the membrane resembles a cell wall in that it is a layer containing fatty substances [lipids] separating components within the lipid nanoparticles from a medium outside the lipid nanoparticles. As described in e.g. Schoenmaker et al. (International Journal of Pharmaceutics, 2021, 601:120586), the membrane of the lipid nanoparticles may be a monolayer or contain one or multiple bilayers.

### Nucleic Acid

The term “nucleic acid” as used herein refers to a polymer containing at least two deoxyribonucleotides or ribonucleotides in either single- or double-stranded form and includes DNA, RNA, and hybrids thereof. DNA may be in the form of antisense molecules, plasmid DNA, cDNA, PCR products, or vectors. RNA may be in the form of small hairpin RNA (shRNA), messenger RNA (mRNA), antisense RNA, miRNA, micRNA, multivalent RNA, dicer substrate RNA or viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid.

### SHORT DESCRIPTION OF DRAWINGS

The present invention will be discussed in more detail below, with reference to the attached drawings, in which:

FIG. 1A shows a CSLM scan;

FIG. 1B shows a schematic figure of amylopectin layered blocks, interpretation of CSLM scan;

FIG. 1C shows a schematic Figure of amylomatrix: amylopectin blocks in amylose matrix;

FIG. 2A shows an amylomatrix product manufactured by injection moulding;

FIG. 2B shows a close-up [2×3 mm] of the product after wetting with moist swab;

FIG. 2C shows a schematic illustration of applying Liquid formulation on Biodegradable Article Surface;

FIG. 2D shows a schematic illustration of an absorption step of liquid formulation;

FIG. 2E shows a schematic illustration of a drying step;

FIG. 3A shows an amylomatrix rod under polarized light;

FIG. 3B shows a picture after 15 minutes to 2 hours in water without enzymes; implant with dissolved amylose, but amylopectin gel persists;

FIG. 3C shows a picture after 2 hours in water with added enzymes [alpha amylase pullulanase];

FIG. 4A shows a picture of an amylomatrix kinetic implant;

FIG. 4B shows a picture of amylomatrix reconstituted to be injected;

FIG. 4C shows a schematic illustration of an Absorption Mechanism;

FIG. 4D shows a schematic illustration of amylomatrix after absorption process;

FIG. 4E shows a schematic illustration of the Release Mechanism after storage;

FIG. 5 shows a graph of size of lipid particles (initial and after storage and release from amylomatrix article);

FIG. 6 shows a graph of mRNA activity;

FIG. 7 shows a graph of thermostability of Bovine Herpes Virus (with/without incorporation in amylomatrix material).

### DETAILED DESCRIPTION OF THE INVENTION

### Cold Chain

The need for refrigeration or freezing during transport, storage and distribution [the “cold chain”] of all types of vaccines is a major drawback, because in practice it appears difficult and often impossible to continuously maintain the cold chain from the vaccine manufacturer until the moment of delivery to the patients, especially in Low and Middle Income Countries [LMIC's].

Therefore, there is a need for an innovative technology to improve vaccine thermostability in order to reach equally all people. Although much effort is put in the development of thermostability of specific vaccines, there is no vaccine thermostability technology established as yet that can stabilize all types of vaccines. Vaccine manufacturers are focussed on the development of vaccines, not on the development of the most stable vaccine formulation. It therefore would be advantageous if a technology could be developed that can be used to stabilize all three types of vaccines [live vaccines, mRNA-LNP's and proteinaceous vaccines] and simultaneously bring the vaccine into a presentation that can directly be delivered to patients, so that vaccine manufacturers do not have to develop such stabilization technology for each vaccine apart.

Therefore, the thermostability challenges are:

1. the “1 month 40° C. challenge”: to improve thermostability of any vaccine platform to a preferred target [e.g. ≥1-2 months at 40° C.] that allows the last stage of the supply chain to occur without cold chain equipment; long term storage may be at 2-8° C. [3-5 years] or at higher temperatures. This relates to vaccines of platform 1: vaccines with proteinaceous constructs.

2. The “3 days 40° C. challenge” to improve thermostability of vaccine platforms that currently require freezing or lyophilization to a minimum target [e.g. ≥12 months at 2-8° C. plus ≥3 days at ° 40 C] that allows them to be distributed via the established cold chain. Mainly aimed at mRNA-LNP vaccines [now frozen] and live viral vaccines [now frozen or lyophilized]. This challenge relates to vaccine platforms 2 [LAV, recombinant Adenovector] and 3 [mainly mRNA-LNP's].

### Logistics

Besides the cold chain, it is in practice a problem to get the vaccines and a variety of different utensilia [syringes, reconstitution needles, injection needles, safety boxes and more] from different places [from the respective manufacturers] at the same time at the sites where these are needed, because of their mere volume and weight; a new technology that not only offers a solution to the thermostability but also to the ease and speed of transport and distribution worldwide would allow equitable access to vaccines anywhere in the world; the solution would be even better if such new technology could offer advantages for the environment, such as reduced energy needed for the manufacturing of the final vaccine presentation, reduced fuel needed for transport, storage and distribution, and for maintaining the cold chain, and reduced wastages and wastes, making the total chain from vaccine manufacturing all through to discarding of waste cheaper, faster and more environmental friendly. This would mean a more equitable access to vaccines worldwide. This is part of the scope of the current invention.

### Explanation of Mechanism of Stabilizing Effect of the Amylomatrix

Without being restricted to theory, the mechanism of stabilisation is further explained as follows: the biologically active constructs are absorbed into the biodegradable material. The biologically active constructs are not homogeneously dispersed within the amylomatrix, but at least partly be arranged in between layers of the amylopectin block (layered phase).

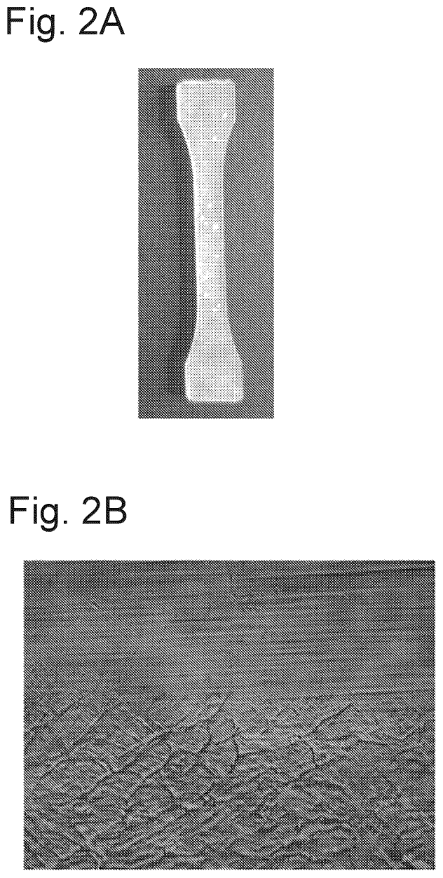

In a first preferred embodiment, a droplet [from <1 μL (1 mcl) to >1 mL (1 cc)] is dropped onto the surface of the shaped article [e.g. a tensile bar], see FIG. 2C, this droplet 25 containing a solute or a suspension [e.g. a biologically active construct]. The water dissolves the upper layer of the amylomatrix, which pulls the water with its solutes [e.g. biologically active construct] in between the amylopectin layers, a bump grows on the surface of the shaped article, see FIG. 2D, arrow 30. During the absorption process, the biologically active constructs are being pulled by hydrogen bonding forces in between the amylopectin layers and there they are being surrounded by the smaller and larger carbohydrate molecules, which stabilize the biologically active construct. When the biologically active construct is caught within the carbohydrates, the pulling of the water molecules, as indicated by arrows H₂O, by the deeper amylopectin layers continues, decreasing the water content at the site of the biologically active construct. After a while, the biologically active constructs are individually surrounded by the amylomatrix with locally a moisture content of sufficiently low concentration as to stabilize the biologically active constructs. As the surrounding carbohydrate molecules immobilize the biologically active constructs, there is no longer intermolecular and intramolecular movements possible at higher temperatures, conveying thermostability. Hereby the distribution of the water within the amylomatrix is getting to an equilibrium within the shaped article. In the final situation all individual biologically active constructs are separated from each other. When kept at ambient temperature and ambient relative humidity, the water at the surface can evaporate from the amylomatrix, as is shown in FIG. 2E by arrows H₂O, thus reducing the water content of the total shaped product. In this form the biologically active construct is thermostable. In another procedure, the shaped article can be frozen within a limited time [several seconds to hours] after the water absorption has been carried out, and freeze dried to get a similar result. In the end, the place where the droplet has touched the surface of the amylomatrix shows a rough surface caused by stilled amylose.

The size of the droplet is not critical and can vary from less than 1 microliter to more than 1 ml (1 cc).

The shaped article can thus be stored at room temperature or higher temperatures. In order to free the biologically active construct from the amylomatrix it suffices to put the shaped article in excess of water. The water will be absorbed again by the amylomatrix until saturation, forming a gel; during this process the soluble carbohydrate molecules such as amylose and smaller carbohydrates as well as any salts including the biologically active constructs will leave the gel with the water, whilst the amylopectin gel itself continues to exist; the gel can be washed out. In the presence of amylase the amylose and the amylopectin gel will degrade and free the biologically active construct faster [about three times faster than without amylase].

Once the amylomatrix has absorbed sufficient water, the layered structure of the original amylopectin layers of the native starch granule has been disrupted by the water and its solution/suspended particles are released.

Thanks to the layered structure of amylopectin, there is a large surface between them; 10 milligrams of amylomatrix has an estimated surface of 400 cm². Any biologically active construct can be arranged on this large surface, separating every single biologically active construct and surrounding it by carbohydrates with no or less reactive hydroxyl-groups; the biologically active constructs are thus packaged and protected like eggs in piled trays.

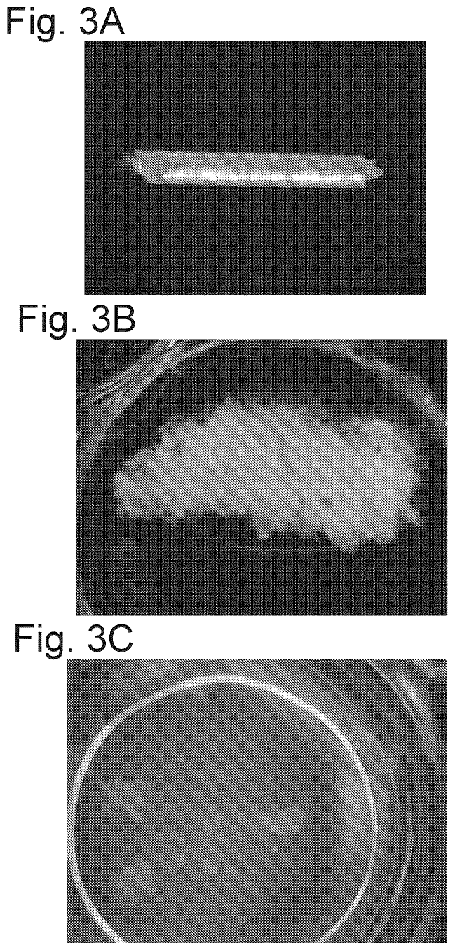

In a second preferred embodiment, the shaped article is a hollow cylinder, with a length of e.g. 15 mm and an outer diameter of 1.16 mm, leaving a cavity of e.g. 0.74 mm in diameter, 12 mm length, having 10 milligram weight and 5 to 7 microliter volume. The cylinder can be closed at one end [with a sharp point] and filled with 1-5 microliter of an aqueous solution containing a biologically active construct. As of the moment of filling the cavity, the inner wall of the cylinder starts absorbing the solution with its solutes 25, as is schematically shown in FIG. 4C. In this example the total weight of the amylomatrix after the absorption process is 17 milligram: 10 mg amylomatrix+7 milligram liquid, which is completely absorbed. The amylomatrix has about 59% dry weight. The amylose in the amylomatrix will partly dissolve and start swelling as indicated by arrows 30.

This absorption process, which is indicated by arrows, can be stopped at any moment by snap freezing, e.g. within 1 second, as taught in prior art for immediate lyophilization after filing the hollow cylinder, to several minutes. For the present invention, it is preferably waited until the aqueous solution has been substantially completely absorbed by the amylomatrix, wherein the cavity is substantially filled by a gel formed by the amylomatrix, as schematically shown in FIG. 4D.

Thereafter, the amylomatrix may be snap frozen by bringing the filled cylinder into intimate contact with a metal carrier which is at a temperature below the eutectic point [mostly in the range of minus 35° C.] until minus 80° C., e.g. minus 50° C.; the amylomatrix including the absorbed aqueous solution then needs only 1 to 2 seconds to freeze; after subsequently freeze drying the dry kinetic implant the biologically active construct has been thermostabilized and can be stored, transported and distributed.

In a next step, at the place and moment of use of the stabilized product the biologically active construct can be released from the shaped article, as schematically shown in FIG. 4E by arrow 40. Additionally, amylose sugars 50 and other components 60 may be released.

One way to use the cylindrical biodegradable article is by kinetically implanting it through the skin in a pain-free way; the speed of SC or IM delivery takes less than 1 millisecond, preventing the generation of more than 1 pain stimulus by the mechanical pain sensors in the skin, so that no pain can be perceived. Once beneath the skin, the amylomatrix absorbs interstitial body fluids [the fluids between the cells], starts swelling and simultaneously the amylose and amylopectin molecules of the amylomatrix are enzymatically degraded within minutes and the biologically active constructs are reconstituted in situ and drained to the lymph nodes. It is noteworthy that the flow of the interstitial fluid is around 10% of its weight every minute. Given the length of the kinetic implant of e.g. about 15 mm, there is a flow of many mLs (cc's) of interstitial fluid between the moment of local delivery and the moment of total dissolution and draining, which is several minutes to more than half an hour later.

### Stabilising Steps

In particular embodiments, the stabilising mechanism of the absorption step and stabilizing process according to the invention comprises:

### Absorption Process:

a. bring liquid formulation, comprising a suspension of lipid particles in water, in contact with matrix of biodegradable material;
b. matrix amylose and smaller carbohydrates dissolves in water, hydrogen bonds attract water molecules with mRNA-LNP's;
c. preferably individualizing mRNA-LNP's within the material of the biodegradable material; keeping LNP's separated from each other inside biodegradable material;
d. Exemplary ratio: biodegradable material matrix: 10 mg versus liquid: 7 mg, [whereby the liquid contains e.g. 10-14 micrograms mRNA-LNP's]. A preferred ratio biodegradable material versus liquid formulation is in the range 10:1 to 1:10 wt/wt. A preferred ratio biodegradable material versus lipid containing composition is in the range of 2000:1 to 20:1 wt/wt.
e. water dissipates deeper into the (dry) matrix, increasing concentration of carbohydrates around LNP's;

Preferably, before the absorption step the biodegradable material is dried to a water content of less than 10 wt. %, preferably less than 5 wt. %.

### After Absorption Process:

f. air dry or vacuum dry, if quantity of water is very small compared to matrix [e.g. <1 weight %-to 20 weight %]; or
g. snap freezing biodegradable material in order to prevent formation of ice crystals, then dry (freeze dry) until less than 5 wt. % water content, preferably less than 3 wt. % water content, more preferably less than 1 wt. % water content

After storage and transport of the biodegradable material (or article) the releasing process is in an embodiment: auto reconstitution in excess of water containing liquid, or FCS [fetal calf serum, 33 IU/l amylase activity].

In a third preferred embodiment, the amylomatrix has the shape of a capsule, e.g. 15 mm×9 mm, with an amylomatrix wall thickness of 3 mm; on the inner side a solution or suspension of the biologically active construct can be introduced; the amylomatrix can absorb the totality of the solution or suspension; optionally the product can be frozen and freeze dried; the capsule thus has a wall that on the outside is original amylomatrix, with an increasing gradient of biologically active constructs towards the inner surface of the capsule; optionally the capsule can be coated for targeting the stabilized biologically active constructs either to the stomach, or the duodenum, or the jejunum at the site of the Peyers' plaques, or the colon. At the targeted site of the gastrointestinal tract, the coating is dissolved and the amylomatrix wall is digested; the stabilised biologically active construct is released to hit the target.

### Elements

### Biodegradable Material

In embodiments, the invention relates to a biodegradable material, which is a solid molecular matrix, consisting of a mixture of two carbohydrate polymers, amylose and amylopectin. Said biodegradable material may further contain smaller carbohydrates, disaccharides and monosaccharides, some lecithin and lipids.

This biodegradable material or processed starch, also referred to as “amylomatrix”, can be manufactured from thermoplastic starch in any shape, such as capsules, powders, films, microneedle patches or small hollow solid dose implants [SDI]. The biodegradable material can be brought into contact with a liquid formulation, which is then absorbed within the biodegradable material. This is in contrast with conventional vaccine stabilization technologies, whereby stabilizers such as trehalose, mannose, amino acids and the like, are added [usually 1% to 10% of dry matter] to the liquid vaccine formulation [“vaccine drug substance”] before further manufacturing. In the present invention it is the biodegradable material itself which absorbs the liquid formulation, thereby at least partly forming a gel phase and remaining a solid carrier for the biologically or pharmaceutically active constructs.

An embodiment of a biodegradable material comprising processed starch according to the invention is known from PCT/NL2008/050120, which is incorporated by reference herein.

The biodegradable material is an excellent starting material for manufacturing biodegradable shaped articles, for example by injection moulding, wherein said biodegradable shaped articles are suitable for delivery of a biologically or pharmaceutically active component in or to a vertebrate, e.g. a mammal. The biodegradable material has a low cytotoxicity.

The biodegradable shaped articles are in particular suitable for parenteral, oral, transdermal, subcutaneous and hypodermic applications.

### Preparation of Biodegradable Material

A process for preparing a biodegradable material according to the invention is, for example, disclosed in PCT/NL2008/050120, which is incorporated by reference herein.

### Water Absorption Property

The biodegradable material preferably has a water absorption property of absorbing at least 50 wt. % of water content, based on the weight of the initial biodegradable material, within 10 minutes of immersion into deionised water.

More preferably, the water absorption property is at least 100 wt. % of water content, based on the weight of the initial biodegradable material, within 10 minutes of immersion into deionised water.

The water absorbance test is described in PCT/NL2008/050120, as is incorporated by reference. It is shown that said biodegradable material absorbs water far more rapidly and in much higher amounts when compared to substantially fully destructurised starch according to EP A 774.975.

### Shaped Article

The shaped article according to the present invention is preferably manufactured by injection moulding, wherein the biodegradable material according to the present invention is subjected to injection moulding at a pressure of about 500 to about 3000 bar (about 50 to about 300 MPa), preferably about 600 to about 2500 bar (about 60 to about 250 MPa), and a temperature of about 100° to about 200° C., preferably about 150° to about 190° C., with residence times of about 5 seconds to about 300 seconds.

Shaped articles, when solubilised at ambient temperature (i.e. about 15° to about 25° C.) in about 50% in DMSO/water, wherein the ratio DMSO:water is 9:1, preferably have a weight average molecular weight of processed amylopectin of about 5.000.000 to about 25.000.000 as determined by MALLS and weight average molecular weight of processed amylose of about 200.000 to about 1.000.000 as determined by GPC-MALLS-RI. In contrast, the weight average molecular weight of amylose in shaped articles made of destructurised starch is much lower than 200.000, e.g. about 120.000, and the weight average molecular weight of amylopectin in shaped articles made of destructurised starch is much lower than 5.000.000, e.g. about 1.000.000. Consequently, although the injection moulding step reduces the weight average molecular weight of amylose and amylopectin also in destructurised starch, the lower values observed in destructurised starch are due to the harsh conditions employed in the preparation of destructurised starch.

The shaped article according to the present invention is in particular suitable for pharmaceutical and nutraceutical purposes and products and for implantation purposes.

Preferably, the shaped article is rod-like, capsule-like, bullet-like, needle-like or tablet-like or has a rod-like, bullet-like, capsule-like, bullet-like, needle-like or tablet-like appearance. It is further preferred according to the present invention that the rod-like, bullet-like or needle-like shaped article has a length:diameter ratio of more than 4, more preferably more than 5, provided that the length of the rod-like or shaped article is between 1 mm to 50 mm. The maximum length:diameter ratio is dependent of various factors like the weight of the rod-like, bullet-like or needle-like shaped article and the application of the rod-like, bullet-like or needle-like shaped article. However, the upper limit of this ratio is about 500, preferably less than about 100, more preferably less than about 75 and most preferably less than about 50. The length of the rod-like or bullet-like shaped article is preferably 2 mm to 25 mm, more preferably 6 mm to 25 mm.

According to the present invention, it may be preferred that the rod-like, bulletlike or needle-like shaped articles have an inner, hollow portion and have an average wall thickness of about 10 μm to about 2500 μm, preferably about 30 μm to about 1500 μm, more preferably about 50 μm to about 500 μm.

Preferably, the rod-like, bullet-like or needle-like shaped articles are provided with a conical tip and a hollow bottom end, although it is obviously possible to provide the hollow rod-like, bullet-like or needle-like shaped articles with a closing means after it is loaded with a substance, for example a biologically active substance as is disclosed in EP A 774.975. In general, hollow rod-like, bullet-like or needle-like shaped articles having an inner, hollow portion are preferred over solid rod-like, bullet-like or needle-like shaped articles.

According to a particular preferred embodiment, the rod-like, bullet-like or needle like shaped article is used as a kinetic implant, said kinetic implant being made from the biodegradable material according to the present invention.

### Kinetic Implant

The kinetic implant is suitable for the parenteral delivery of biologically active substances. Parenteral delivery includes delivery by injection or infusion which may be intravenous, intraarterial, intramuscular, intracardiac, subcutaneous, intradermal, intrathecal, transdermal, and transmucosal. Preferably, the kinetic implant is used for intramuscular, subcutaneous and transdermal delivery.

The weight of the kinetic implant is preferably such that the kinetic implant can be provided with an amount of kinetic energy in the range of about 0.1 to about 10 J, preferably about 0.2 to about 5 J. This implies that, if the kinetic implant is accelerated to a velocity comparable to the sound velocity (in dry air at about 20° C., the sound velocity is about 340 m/s), the minimum weight is about 1 mg whereas the maximum weight is about 180 mg.

However, for human applications, it is in particular preferred that the kinetic energy (based on a velocity of about 340 m/s) of the kinetic implant is in the range of 0.1 to 5 J, preferably 0.1 to 3 J. If higher kinetic energies (based on a velocity of about 340 m/s) are employed, the kinetic implant becomes too awkward for human application.

### Biologically Active Constructs

The product according to the invention contains a biologically or pharmaceutically active construct. The term “biologically active construct” includes any construct that has a biological effect or response, e.g. a therapeutic, a prophylactic, a probiotic or an immunising effect, when it is administered to a living organism (in particular a vertebrate) or when a living organism is exposed in some way to the biologically active construct. The biologically active construct may also be referred to as a pharmaceutically active construct.

Consequently, the term “biologically or pharmaceutically active constructs” includes pharmaceutical agents, therapeutic agents and prophylactic agents. Suitable examples of pharmaceutical agents are anti-inflammatory drugs, analgesics, antiarthritic drugs, antispasmodics, antidepressants, antipsychotics, tranquilizers, antianxiety drugs, narcotic antagonists, antiparkinsonism agents, cholinergic agonists, chemotherapeutic drugs, immunosuppressive agents, antiviral agents, antibiotic agents, appetite suppressants, antiemetics, anticholinergics, antihistaminics, antimigraine agents, coronary, cerebral or peripheral vasodilators, hormonal agents, contraceptives, antithrombotic agents, diuretics, antihypertensive agents, cardiovascular drugs and opioids. Suitable examples of therapeutic or prophylactic agents are subcellular compositions, cells, viruses, molecules including lipids, organic compounds, proteins and (poly) peptides (synthetic and natural), peptide mimetics, hormones (peptide, steroid and corticosteroid), D and L amino acid polymers, oligosaccharides, polysaccharides, nucleotides, oligonucleotides and nucleic acids, including DNA and RNA, protein nucleic acid hybrids.

Suitable examples of proteins and (poly) peptides are enzymes, biopharmaceuticals, growth hormones, growth factors, insulin, monoclonal antibodies, interferons, interleukins and cytokines. Suitable examples of prophylactic agents are immunogens such as vaccines, e.g. live and attenuated viruses, nucleotide vectors encoding antigens, antigens.

Vaccines may be produced by molecular biology techniques to produce recombinant peptides or fusion proteins containing one or more portions of a protein derived from a pathogen. The biologically active substance may be derived from natural sources or may be made by recombinant or synthetic techniques.

### Lipid Containing Composition Comprising Lipid Nanoparticles

In various embodiments, the lipid nanoparticles have a mean diameter of from about 10 nm to about 300 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic. The mean diameter of the lipid nanoparticles may be determined using dynamic light scattering measurements, optionally using a Zetasizer Pro Red Light Scattering System, Advance Series (Malvern Analytics).

### Lipids

The term “polymer conjugated lipid” refers to a molecule comprising both a lipid portion and a polymer portion. An example of a hydrophilic polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-DMG) and the like.

The term “neutral lipid” refers to any of a number of lipid species that exist either in an uncharged or neutral zwitterionic form at a selected pH. At physiological pH, such lipids include, but are not limited to, phosphatidylcholines such as 1,2-Distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-Palmitoyl-2-oleoyl-sn-glycero-3-phosphocholine (POPC), 1,2-dioleoyl-sn-glycero-3-phosphocholine (DOPC), phosphatidylethanolamines such as 1,2-Dioleoyl-sn-glycero-3-phosphoethanolamine (DOPE), sphingomyelins (SM), ceramides, steroids such as sterols and their derivatives. Neutral lipids may be synthetic or naturally derived.

The term “charged lipid” refers to any of a number of lipid species that exist in either a positively charged or negatively charged form independent of the pH within a useful physiological range e.g. pH 3 to pH 9. Charged lipids may be synthetic or naturally derived. Examples of charged lipids include phosphatidylserines, phosphatidic acids, phosphatidylglycerols, phosphatidylinositols, sterol hemisuccinates, dialkyl trimethylammonium-propanes, (e.g. di-oleyl-3-trimethylammonium propane (DOTAP), 1,2-di-O-octadecenyl-3-trimethylammonium propane (DOTMA)), dialkyl dimethylaminopropanes, ethyl phosphocholines, dimethylaminoethane carbamoyl sterols (e.g. 3ß-[N—(N′,N′-dimethylaminoethane)-carbamoyl] cholesterol (DC-Chol)).

In various embodiments, the polymer conjugated lipid is a pegylated lipid. For example, some embodiments include a pegylated diacylglycerol (PEG-DAG) such as 1-(monomethoxy-polyethyleneglycol)-2,3-dimyristoylglycerol (PEG-DMG), a pegylated phosphatidylethanolamine (PEG-PE), a PEG succinate diacylglycerol (PEG-S-DAG) such as 4-O-(2′,3′-di(tetradecanoyloxy)propyl-1-O-(ω-methoxy(polyethoxy)ethyl)butanedioate (PEG-S-DMG), a pegylated ceramide (PEG-cer), or a PEG dialkoxypropylcarbamate such as ω-methoxy (polyethoxy)ethyl-N-(2,3-di(tetradecanoxy)propyl)carbamate or 2,3-di(tetradecanoxy)propyl-N-(ω-methoxy(polyethoxy)ethyl)carbamate.

### Composition Comprising Processed Starch and a Lipid Containing Composition

Also provided herein is a composition comprising processed starch and a lipid containing composition comprising lipid nanoparticles and a pharmaceutically active agent. The processed starch is as defined above.

The composition may comprise the processed starch and the lipid containing composition in a weight ratio of lipid containing composition (calculated based on the total weight of the lipid nanoparticles and the pharmaceutically active agent) in a weight ratio of processed starch to lipid containing composition of 50:1 to 40000:1, in particular of 75:1 to 30000:1, more in particular 100:1 to 20000:1, more in particular 2000:1 to 10000:1.

The pharmaceutically active agent may be a nucleic acid. The nucleic acid may be selected from the group consisting of ribonucleic acid (RNA) and desoxyribonucleic acid (DNA), in particular selected from the group consisting of messenger RNA, silencing RNA and antisense RNA.

The composition may have a water content of less than 5 wt. %, preferably less than 3 wt. %, more preferably less than 1 wt. %. Such a water content is advantageous for storing and transporting the composition, or products containing the composition, outside the cold chain.

The lipid nanoparticles may have a defined particle size. In some embodiments, at least 50% of the lipid nanoparticles have a particle size in the range of 10 nm-200 nm as determined by dynamic light scattering, preferably at least 80%, more preferably at least 90%, wherein the percentage is a number %. The lipid nanoparticles may have an average particle size in the range of 50 to 200 nm, in particular in the range of 60 to 150 nm, more in particular in the range of 90 to 120 nm, as measured by dynamic light scattering (e.g. using a Zetasizer Pro Red Light Scattering System, Advance Series (Malvern Analytics)). It has been found that the particle size of the lipid nanoparticles is surprisingly stable, even at high temperatures.

The lipid nanoparticles may comprise one or more neutral lipids selected from 1,2-distearoyl-sn-glycero-3-phosphocholine (DSPC), 1,2-dipalmitoyl-sn-glycero-3-phosphocholine (DPPC), 1,2-dimyristoyl-sn-glycero-3-phosphocholine (DMPC), 1,2-dioleoyl-sn-glycero-3-phosphocholine (DOPC), 1-palmitoyl-2-oleoyl-sn-glycero-3-phosphocholine (POPC), 1,2-dioleoyl-sn-glycero-3-phosphoethanolamine (DOPE) and sphingomyelin (SM).

The composition may further comprise a stabilizing component stabilizing the pharmaceutically active agent. The stabilizing component may be selected from the group consisting of monosaccharides, disaccharides, trisaccharides, oligosaccharides, polysaccharides, glycoproteins, proteoglycans, peptidoglycans, glycolipids, lipopolysaccharides, and phosphonomannans, in particular a disaccharide, more in particular a non-reducing disaccharide, more in particular a trehalose (α-D-glucopyranosyl-α-D-glucopyranoside). Oligo- and polysaccharides used as stabilizing agent may be linear or branched.

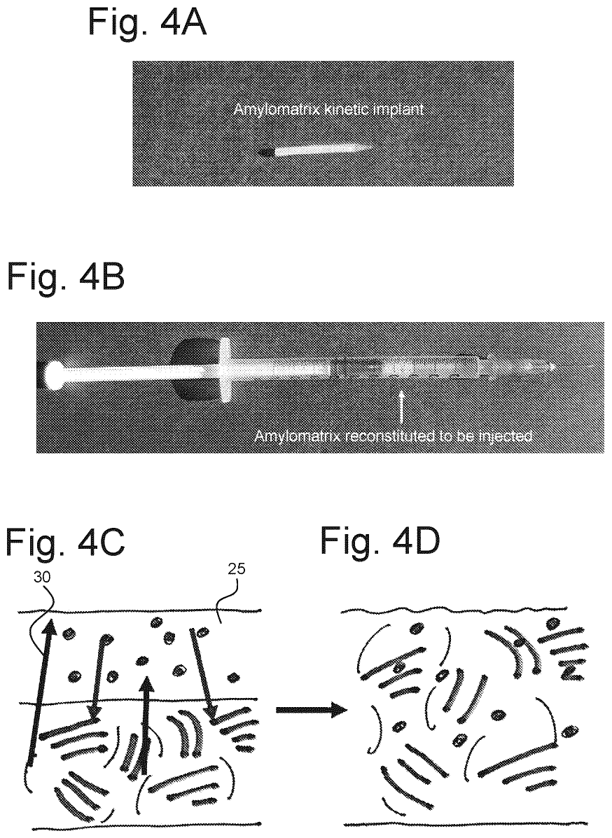

Stabilizing lipid nanoparticles, in particular mRNA-LNPs, has significant and surprising advantages, as shown in Experiments 1-7. For example, the liquid nanoparticles do not fuse, aggregate or disintegrate when stabilized on a processed starch (see, Experiment 6 and FIG. 5), even after storage at elevated temperatures for multiple weeks. This is advantageous, as fusion, aggregation and disintegration of liquid nanoparticles are associated with reduced efficacy of the pharmaceutically active agent, in particular when the pharmaceutically active agent is a nucleic acid.

The composition defined above may be for use in therapy or prophylaxis. The composition for use may be administered to the subject (e.g., a human or an animal) intramuscularly or subcutaneously. This leads to significant practical advantages, as reconstitution of a composition defined above (especially one with a water content of less than 5.0 wt. %) that has been administered intramuscularly or subcutaneously takes place in situ. Accordingly, no separate reconstitution step (e.g. in a syringe) is required.

### Process for Storing Biologically Active Constructs

A process for storing biologically active constructs in a biodegradable material, the method comprising the steps:
a) providing a biodegradable material, wherein the biodegradable material comprises or consists essentially of a processed starch;
b) providing biologically active constructs, preferably providing a liquid formulation comprising the biologically active constructs and a liquid carrier;
c) absorbing said biologically active constructs into the biodegradable material; and
d) storing the biologically active constructs in the biodegradable material at a storage temperature from −80° C. to 80° C. for a period of at least 1 day.

The liquid carrier used in the process may comprise water. It may, for example, be a buffered aqueous solution, such as phosphate-buffered saline.

The process comprises an absorbing step. This absorbing step may comprising absorbing a stabilizing component into the biodegradable material comprising the biologically active construct. The stabilizing component stabilizes the biologically active construct and may be selected from the group consisting of monosaccharides, disaccharides, trisaccharides, oligosaccharides, polysaccharides, glycoproteins, proteoglycans, peptidoglycans, glycolipids, lipopolysaccharides, and phosphonomannans, in particular a disaccharide, more in particular a non-reducing disaccharide, more in particular a trehalose (α-D-glucopyranosyl-α-D-glucopyranoside).

Following the absorbing step, the process may further comprise a step of cooling the biodegradable material comprising the biologically active constructs. The cooling preferably comprises cooling the biodegradable material to a temperature of −70° C. to −30° C. It is preferred that the cooling is performed rapidly to avoid the formation of crystals within the biodegradable material. Accordingly, the cooling is preferably performed within a period of 0.1 seconds to 30 seconds, preferably within 10 seconds, more preferably within 5 seconds. This can be achieved by subjecting the biodegradable material comprising the biologically active material to a temperature of e.g. −78° C. or less.

The cooling is preferably commenced within 0.1 to 180 seconds following the start of the absorbing step, in particular within 0.1 to 60 seconds, more in particular within 1.0 to 30 seconds. This is particularly advantageous when the biodegradable material is provided as a rod-like, bullet-like, or needle-like shaped article (e.g. a kinetic implant) having an inner, hollow portion and an average wall thickness of 10 to 2500 μm, in particular 30 to 1500 μm, more in particular 50 to 500 μm, as the shape of the biodegradable material is then maintained. Longer periods between the start of the absorbing and the cooling time (i.e., the absorbing period) may lead to partial deformation of a biodegradable material provided as a shaped article, which, in turn, may reduce the suitability of the shaped article as e.g. a kinetic implant.

The cooling may also be commenced within 45 to 600 seconds following the start of the absorbing step, in particular within 60 to 300 seconds, more in particular within 90 to 180 seconds. This is particularly advantageous, as longer contacting times allow for better penetration of the biologically active constructs into the biodegradable material. Better penetration of the biologically active constructs into the biodegradable material, in turn, results in improved thermostability. Accordingly, a longer absorbing period can be advantageous if the biologically active construct is particularly thermosensitive. It may be preferred that, when a longer absorbing period is used, the biodegradable material is provided in the form of a tablet or a capsule.

The process may further comprise a step of drying the biodegradable material comprising the biologically active constructs to a water content of less than 5 wt. %, preferably less than 3 wt. %, more preferably less than 1 wt. %. This drying may be done using methods commonly known in the art, such as using a freeze dryer. In some embodiments (e.g. when the biologically active construct is a virus, virosome or virus-like particle), the drying may comprise drying the biodegradable material and the biologically active constructs to a water content of 0.5 to 5.0 wt. %, preferably 1.0 to 4.0 wt. %, more preferably 2.0 to 3.0 wt. %.

The biologically active construct may, for example, be a virus, a virus-like particle, or virosome. The biologically active construct may be a virus selected from one or more of the group consisting of Herpesviridae, Adenoviridae, Bunyaviridae, Filoviridae, Rhabdoviridae, Retroviridae, Reoviridae, Coronaviridae, Orthomyxoviridae, Paramyxoviridae, Togaviridae, Papillomaviridae, Poxviridae and Flaviviridae. The virus may be an attenuated virus, an inactivated virus, or a split virus, preferably an attenuated virus or an inactivated virus. The virus may also be a genetically modified virus from the family Adenoviridae. The virus may also be a combination of viruses, in particular a combination of viruses from the family of Togaviridae and from the family of Paramyxoviridae.

In some embodiments of the process as described in this section, the biologically active construct does not comprise lipid nanoparticles.

### Composition Comprising a Processed Starch and a Virus

Also provided herein is a composition comprising (a) a processed starch (as defined above) and (b) a virus selected from one or more of the group consisting of Herpesviridae (in particular, a Varicellovirus, more in particular bovine alphaherpesvirus 1), Adenoviridae, Bunyaviridae, Filoviridae, Rhabdoviridae, Retroviridae, Reoviridae, Coronaviridae, Orthomyxoviridae, Paramyxoviridae, Togaviridae, Papillomaviridae, Poxviridae and Flaviviridae.

The composition may comprise 5.0 to 99.9 wt. % of processed starch, in particular 10 to 99 wt. %, more in particular 25 to 90 wt. %.

The virus may be an attenuated virus, an inactivated virus or a split virus, preferably an attenuated virus or an inactivated virus. The virus may also be a genetically modified virus, e.g. a genetically modified virus from the family Adenoviridae. The virus may also be a combination of viruses, in particular a combination of viruses from the family of Togaviridae and from the family of Paramyxoviridae.

The composition may have a water content of 0.5 to 5.0 wt. %, preferably 1.0 to 4.0 wt. %, more preferably 2.0 to 3.0 wt. %. Such a water content is advantageous for storing and transporting the composition, or products containing the composition, outside the cold chain.

The lipid nanoparticles may comprise one or more neutral lipids selected from 1,2-distearoyl-sn-glycero-3-phosphocholine (DSPC), 1,2-dipalmitoyl-sn-glycero-3-phosphocholine (DPPC), 1,2-dimyristoyl-sn-glycero-3-phosphocholine (DMPC), 1,2-dioleoyl-sn-glycero-3-phosphocholine (DOPC), 1-palmitoyl-2-oleoyl-sn-glycero-3-phosphocholine (POPC), 1,2-dioleoyl-sn-glycero-3-phosphoethanolamine (DOPE) and sphingomyelin (SM).

The composition may further comprise a stabilizing component stabilizing the pharmaceutically active agent. The stabilizing component may be selected from the group consisting of monosaccharides, disaccharides, trisaccharides, oligosaccharides, polysaccharides, glycoproteins, proteoglycans, peptidoglycans, glycolipids, lipopolysaccharides, and phosphonomannans, in particular a disaccharide, more in particular a non-reducing disaccharide, more in particular a trehalose (α-D-glucopyranosyl-α-D-glucopyranoside). Oligo- and polysaccharides used as stabilizing agent may be linear or branched.

The composition may be free from lipid nanoparticles.

As shown in Experiment 8 below, viruses can successfully be stored on a processed starch, even at elevated temperatures. More specifically, Experiment 8 demonstrates that Bovine Herpes Virus 1 (BHV1), a member of the family Herpesviridae, can be stored at 37° C. for several days with almost no reduction of BHV1 titer without the addition of stabilizing agents. Remarkably, the BHV1 titer was still 98.5% of the original BHV1 titer after storage at 37° C. for 28 days. This suggests that compositions comprising a processed starch and a virus may be more stable than compositions comprising a processed starch and an antigen (such as those described in WO 2008/105663, Example 7).

The composition defined above may be for use in the treatment or prevention (preferably, for use in the prevention) of an infection with a virus selected from one or more of the group consisting of Herpesviridae (in particular, a Varicellovirus, more in particular bovine alphaherpesvirus 1), Adenoviridae, Bunyaviridae, Filoviridae, Rhabdoviridae, Retroviridae, Reoviridae, Coronaviridae, Orthomyxoviridae, Paramyxoviridae, Togaviridae, Papillomaviridae, Poxviridae and Flaviviridae. In some embodiments, the composition may be administered to a subject (e.g., a human or an animal, in particular cattle) intramuscularly or subcutaneously. This leads to significant practical advantages, as reconstitution of a composition defined above (especially one with a water content of 0.5 to 5.0 wt. %) that has been administered intramuscularly or subcutaneously takes place in situ. Accordingly, no separate reconstitution step (e.g. in a syringe) is required.

### EXPERIMENTAL

### Examples

### Experiment 1: Microscopy of the Structure of the Amylomatrix

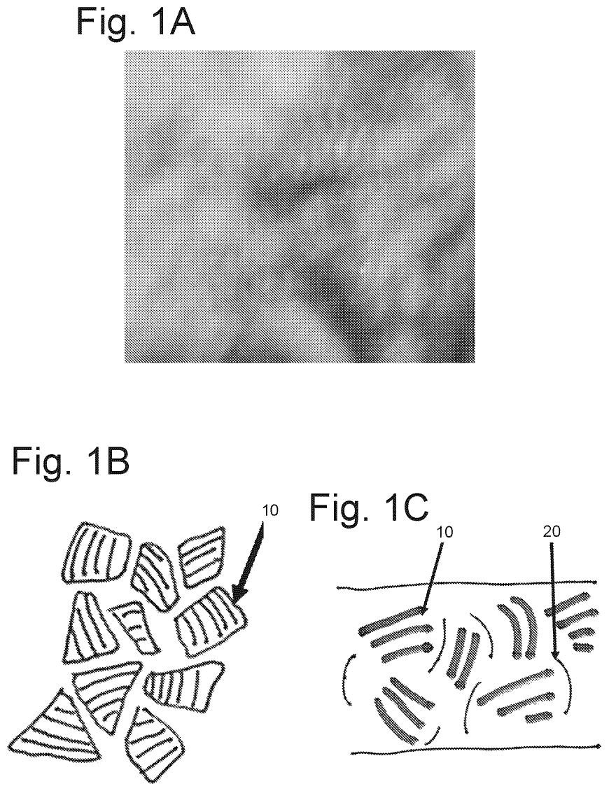

See FIG. 1A, which shows a CLSM image of a solid amylomatrix, rod shaped, 10 mm long, 1 mm Ø, produced by injection moulding, dry, under confocal scanning microscope, autofluorescent amylopectin, wavelength±570 nm, picture 12 um×10 um, 42 layers scanned with CSLM [Confocal Scanning Microscope], every scan 1 um deeper, reconstructed to 3D image, showing amylopectin layers about 200 nm thick and corresponding with the original growth rings of the starch granule; distance between the amylopectin layers is about 50 to 150 nm by measurement by comparing to total size [12 μm×10 μm].

See FIG. 1B, which shows a schematic Figure of a amylomatrix model: pieces of starch granules, consisting of amylopectin layers 10, spaces in between filled with [transparent] amylose that has been leaked, cementing the layered amylopectin blocks 10. Starch granules were in the presence of water broken by extrusion into pieces in a controlled manner as not to fully destructurize the granules [as is the case in thermoplastic starch]; breaking the starch in a controlled manner is comparable to the production of pregelatinized starch. Pregelatinized starch [See “Starch Chemistry and Technology”] is starch that rapidly forms a gel when wetted. Amylomatrix can thus be considered as a pregelatinized state of TPS, or “gel grade TPS”.

See FIG. 1C, which derived from FIG. 1B, whereby the contours of the layered amylopectin blocks 10 were omitted and amylose molecules 20 drawn as curved lines in between the layered amylopectin blocks 10, cementing the layered amylopectin blocks; in between the layered amylopectin blocks and the amylose molecules a range of smaller carbohydrates [such as dextrines] are present. In this patent this is the proposed model for the amylomatrix.

### Experiment 2: Superficial Inoculation of Surface of Amylomatrix

See FIG. 2A, showing an amylomatrix product manufactured by injection moulding; the product measures 7 cm long, 5 mm wide and 2 mm thick.

See FIG. 2B, which shows a close-up [2×3 mm] of the product after wetting with moist swab; upper half: original surface; bottom half: surface of the amylomatrix after drying at ambient temperature at ambient air.

The solid product, produced by injection moulding, was wetted by using a moist swab [lower half of the picture]; the water from the swab was absorbed by the surface of the product, whereby the amylomatrix dissolves in the water; due to increase of volume, the surface of the product raises slightly and becomes irregular; partly the water was drawn deeper into the amylomatrix and partly the water evaporated from the surface into the ambient air; after about 10 to 20 minutes, the irregular surface was solidified. Any solutes and particles that were present in the moist have thus been absorbed and dried inside the amylomatrix.

### Experiment 3: Amylomatrix Pulls Aqueous Solution Inside/Amylomatrix Absorbing Solution

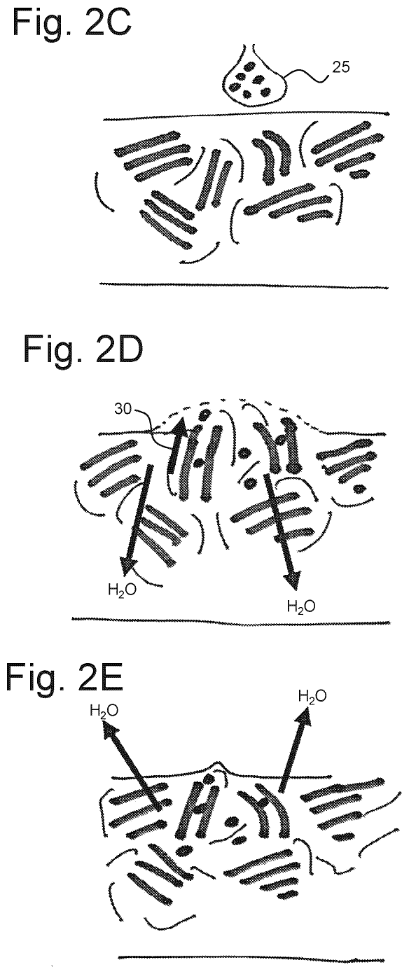

See FIG. 3A, which shows an amylomatrix rod. Said solid rod was produced by injection moulding, 16 mm long, 1.16 mm Ø, weight about 10 mg, showing polarization; given the amylopectin layers being about 250 nm thick, the total surface of amylopectin layers in this rod can be estimated: 250 nm thick layers piling up to 10 mm [=10,000,000 nm] gives 40,000 layers; given the surface of the cross section of the rod being π. r²=3.14. [0.58]²=about 0.01 cm², the total surface of amylopectin layers in 10 mg amylomatrix is about 40,000×0.01=400 cm². It is believed that biologically active ingredients may be at least partly arranged in between the amylopectin layers in e.g. mono layers as eggs in trays; it is believed that this allows the separation of all individual biologically active ingredients; the space in between the amylopectin layers being in the range of 50 to 500 nm allows biologically active ingredients to be arranged such as e.g. but not limited to viruses and mRNA-LNP's.

The solid rod of FIG. 3A was added to an excess of water, see FIG. 3B; the water was absorbed by the amylomatrix, thereby dissolving the amylose; the amylopectin layers pull the water in between their layers thanks to the broken hydrogen bonds, whereby the amylopectin layers widen maximally without dissolving, but instead forming a non-soluble and non-compressible gel; any solutes or suspended particles in between the amylomatyrix layers are released. In this way e.g. particles like viruses [live, attenuated or inactivated] and mRNA-LNP's can be released.

If the solid rod of FIG. 3A was added to an excess of water containing amylase [alpha amylase pullulanase], not only the dissolved amylose is enzymatically hydrolysed, but also the amylopectin gel, see FIG. 3C; the result is a [nearly] completely degraded and dissolved amylomatrix.

In general, when the amylomatrix comes into contact with an excess of water, it will absorb this water until about 10 to 20 times its own weight. When the amylomatrix is delivered in subcutaneous or muscular tissues, the absorbed water will be the interstitial fluid; this fluid contains serum amylase, which breaks down amylose and amylopectin molecules very fast: as soon as an amylose or amylopectin molecule comes into contact with serum amylase, the breakdown is fast enough to prevent any amylose molecule or amylopectin molecule to come into circulation.

### Experiment 4: Incorporation of a Biologically Active Construct in a Hollow Kinetic Implant and the Release from it

A hollow cylindrical amylomatrix was manufactured by injection moulding, 12 mm long 1.2 mm diameter, weighing 10 mg, see FIG. 4A; the cavity was filled with 5 μl water containing a suspension of particles. From the inside of the cylinder the amylomatrix starts absorbing the water with the suspended particles, which were then pulled in between the amylopectin layers and in between the amylose molecules; the total volume of suspension was no more than about 50% of the weight of the amylomatrix; total absorption time was about 2 minutes, after which all of the suspension was absorbed; after absorption of the suspension into the amylomatrix, the absorption process was stopped by sudden freezing, id est by freezing within maximum 5 seconds [preferably within 2 or 1 second, snap freezing]; this is achieved by putting the cylindrical amylomatrix into a metal holder, narrowly fitting the amylomatrix; the metal holder was precooled at a temperature of minus 50 to minus 70 degrees Celsius; it can be observed that the freezing step takes this very short time, called “snap freezing”, in contrast to the standard freezing process taking typically several minutes to more than half an hour]. After freezing the particles containing amylomatrix is freeze dried using standard freeze drying equipment and standard drying trajectories; typically such trajectory starts at minus 50° C. to minus 35° C. with linearly increasing temperatures up until plus e.g. 20° C. after e.g. 24 hours. After freeze drying, the amylomatrix is taken out of the metal holder, for [e.g. long term] storage. The particles are positioned partly in between the amylopectin layers and partly surrounded by amylose molecules; every particle is thus immobilized by chemically inert material, conferring thermostability.

After storage and transport anywhere and on any desired moment, the amylomatrix containing the immobilized particles is put into an excess of water, e.g. 1 cc in a syringe, see FIG. 4B; the water dissolves the amylose molecules and is drawn in between the amylopectin layers; the distance between the amylopectin grows with factor 5 to 20 [depending on the quantity of excess of water added] to form a gel; the pores within the gel grow concomitantly thereby giving space to the particles that are released from the gel to form a suspension; the suspension then can be injected into the body from the syringe using a standard injection needle.

Alternatively, the amylomatrix can be kinetically implanted into the body, where in the presence of amylase [e.g. in human tissues] the amylopectin is hydrolysed yielding glucose molecules; enzymatic hydrolysis is very fast [several minutes]; the result is that the particles are released into the body 2 to 10 times faster than when no amylase is present.

### Experiment 5: Amylomatrix Incorporating Biologically, e.g. mRNA-LNP's

Preparation of mRNA-LNP's in Amylomatrix.

mRNA-LNP's size 100 nanometer were produced as described in literature [reference: “Lipid Nanoparticle Systems for enabling gene therapies” by Pieter R. Cullis et al., 2017] using microfluidics with ionizable cationic lipids, cholesterol type of molecule, DSPE-PEG-2000 and DSPC; two types of mRNA were used: either fluorescently labelled with AlexaFluor 647 or coding for luciferase; 120 hollow mini implants [small hollow cylindrical tubes [16 mm×1.16 mm, inner cavity diameter 0.7 mm] were produced of the amylomatrix as described in literature; each implant was filled with 5 microliter of a suspension of either type of mRNA-LNP's, containing in total 10 micrograms of mRNA-LNP's; the amylomatrix absorbed the suspension during at least half a minute, absorbing the totality of the mRNA-LNP's within its amylomatrix; after 30 seconds to 60 seconds the mini implants were brought into intimate contact with an aluminium block at minus 50° C. to minus 60° C. and the amylomatrix froze within maximum 2 seconds; thanks to the amylomatrix having absorbed the water suspension, the concentration of the water at the level of the mRNA-LNP has dramatically decreased until less than 50% whilst the concentration of the carbohydrates is very high; this in combination with the snap freeze procedure prevents water to crystallize into large crystals, thus protecting the LNP's. The LNP's are pulled in between the amylopectin layers [like eggs in trays] and completely separated one from each other; each individual LNP is completely packaged in between the carbohydrates, in between the amylopectin layers; the amylopectin blocks having the same size range as the LNP's [100-250 nm] and being covered with glucose-chains of average 15 Glu-moieties, this means that every single LNP is fully encapsulated by the amylopectin blocks with in between amylose chains and smaller carbohydrates, mainly amylose; after snap freezing, the mini implants are freeze dried under vacuum for 24 hours, starting with a temperature of minus 50° C., raising temperature until +20° C. after 24 hours until a moisture content of less than 5%, preferably less than 3%; the final product consists of an amylomatrix with layers of amylopectin blocks, with LNP's in between and cemented with amylose molecules; the spaces in between are filled with smaller carbohydrate molecules. Given the water content of the amylomatrix after absorption of the mRNA-LNP suspension is in the range of [less then] 50%, the freeze drying process dries the final product leaving small pores, such as in the range of, or smaller than 100 nm. The final products were stored at 2° C.-8° C. during 40 days, at 20° C. during 9 days, at 37° C. during 9 days and at 45° C. during 3 days.

### Experiment 5: mRNA-LNP's Release from Amylomatrix

Prior to release, the amylomatrix were stored dry for few days at 4° C. in closed vials. LNPs containing fluorescently-labelled mRNA (AlexaFluor 647) were liberated from the amylomatrix by incubation into 100% FCS (foetal calf serum) as mimic of bodily fluids, the amylase enzymatic activity was 33 IU/l. A single amylomatrix was incubated in 1 ml of FCS at 37° C., with gentle agitation.

Samples (25 μl) were taken at indicated timepoints and centrifuged at 350 g for 3 minutes at room temperature. Such centrifugation selectively removes macroscopic particles, but leaves all nanoparticles in solution. The supernatant was retrieved and measured in optically-isolated wells of a 384-well plate on a plate reader (iD3, Molecular Devices for DLS dynamic Light Scattering) with auto-optimization settings and dynamic measurement sensitivity as recommended by the manufacturer.

As can be appreciated from the two Tables, see Tables 5A-5B, we can conclude that the amylomatrix shows rapid swelling and loss of structural integrity to form a gel that fully releases the LNP-encapsulated mRNA (AF647 material) within 60 minutes. From the graphs it is concluded that 90% of the mRNA-LNP's that were incorporated into the amylomatrix were retrieved after reconstitution in FCS [fetal calf serum] at 37° C.

TABLE 5A
speed of release of LNP's from the amylomatrix
after reconstitution [hours]

| Hours | % release |
|---|---|
| 0 | 0 |
| 1 | 92 |
| 2 | 94 |
| 3 | 98 |
| 6 | 98 |
| 24 | 100 |

TABLE 5B
speed of release of LNP's from the amylomatrix
after reconstitution [minutes]

| Minutes | % release |
|---|---|
| 0 | 0 |
| 3 | 2 |
| 6 | 8 |
| 10 | 3 |
| 15 | 55 |
| 30 | 70 |
| 60 | 100 |

Table 5C shows the release of mRNA-LNP's using water-based swelling (without enzymes):

TABLE 5C
release of mRNA-LNP's using water-based swelling

| | |
|---|---|
| Time | % release |

| | | |
|---|---|---|
| 0 | minutes | 0 |
| 3 | minutes | 1 |
| 6 | minutes | 4 |
| 10 | minutes | 10 |
| 15 | minutes | 18 |
| 30 | minutes | 30 |
| 1 | hour | 55 |
| 3 | hour | 90 |
| 24 | hour | 100 |

As can be appreciated from these results in Tables 5A-5C, it can be concluded that the release from the amylomatrix is via water-based swelling, release rate is ˜2-3× slower than with enzymes (90% release is achieved in part of samples after 180 min, whereas all samples showed 90% released in 60 min in 100% FCS). The release time seems largely unaffected by storage temperature, see Table 5D, indicating that also the chosen PEG-ylated lipid conjugates in the outer layer of the LNP's remain intact and active over time and at elevated temperatures. The amylomatrix has stabilized the LNP's even at 45° C.

TABLE 5D
release of mRNA-LNP's using water-based swelling (without
enzymes) in relative, indicative percentages after different
storage temperatures and different periods of time.

| | | |
|---|---|---|
| storage temp. | days stored | % release |

| | | | |
|---|---|---|---|
| 4° | C. | 60 | 95 ± 30 |
| 20° | C. | 9 | 80 ± 15 |
| 35° | C. | 9 | 70 ± 20 |
| 45° | C. | 3 | 100 ± 15 |

### Experiment 6-LNP Size Distribution

LNPs were released from amylomatrix using Phosphate-buffered Saline (PBS) without serum (serum components can form a protein-corona on the nanoparticles, and affect size measurements), hence without amylase. Prior to release of the LNPs, the amylomatrix were stored for multiple weeks at indicated temperatures (4° C., 20° C. [room temperature], 35° C., 45° C.). After release for 12 h, the amylomatrix fragments were removed by centrifugation at 350 g for 3 minutes at room temperature. Such centrifugation selectively removes macroscopic particles, but leaves the majority of micro-sized particles and all nanoparticles in solution. The undiluted solution containing the released LNPS was subjected to dynamic light scattering (DLS, Malvern Analytics) under standard manufacturer recommended settings. The size-distribution of the nanoparticles was investigated for each of the previously indicated storage temperatures.

As can be appreciated from the graph, see FIG. 5, it is concluded that storage for multiple weeks did not change LNP size significantly [LNPs were ˜60 nm before storage]; it can also be concluded that storage temperature did not change LNP size; all samples are identical in size distribution; and that processing of LNPs during incorporation in amylomatrix did not induce major structural damage; otherwise average size and size-distribution would have shown changes. It can be concluded that the absorption of the LNP's by the amylomatrix, followed by the processes of freezing, vacuum drying, storing at different temperatures, and reconstitution do not affect the LNP's, hence that the amylomatrix stabilizes the LNP's.

### Discussion/Conclusion

Mini amylomatrix implants containing the mRNA-LNP's were reconstituted in water at 37° C.; the amylomatrix started to absorb immediately the water in quantities about 10 to 20 times its own weight; during this process the layers of amylopectin blocks widen, giving a gel, but do not dissolve and the soluble carbohydrates dissolve. During this gelation and dissolving process, the LNP's are coming back into suspension and thanks to gentle swirling the mRNA-LNP's leave also the gel. Measuring the numbers of mRNA-LNP's reveals that the mRNA-LNP's that had been incorporated within the amylomatrix could be recovered within 1 hour [in the presence of amylase this process could be accelerated three times]. It is surprising that the 90% recovery was found in amylomatrix mini implants independent from the temperature at which they were stored. The surprising finding is that the LNP's are stable even at 20° C. and 35° C. during 9 days and at 45° C. during 3 days. This fulfills a “3 days 40° C. challenge” for the LNP-part of the LNP's.

The measurement of the size of the mRNA-LNP's reveal that their size has not changed, and hence this experiment proves that the amylomatrix prevents the fusion, aggregation and disintegration of the mRNA-LNP's, leaving the original LNP's intact. Overall conclusion is that the amylomatrix has stabilized 90% of the mRNA-LNP's, and this even at 45° C.

### Experiment 7: Luciferase Expression in Cell Culture

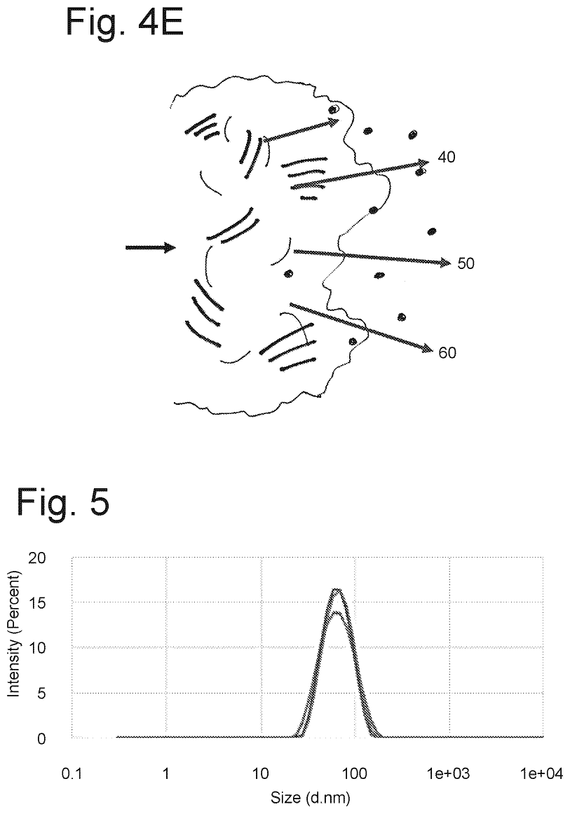

LNPs containing non-fluorescently-labelled mRNA were released from amylomatrix by incubation into 100% FCS (foetal calf serum) as mimic of bodily fluids with maximal enzymatic amylase activity [33 IU/l]. A single amylomatrix was incubated in 1 ml of cell culture medium with FCS at 37° C., with gentle agitation, for 30 min. The solution was centrifuged at 350 g, for 3 minutes at room temperature. The supernatant, containing the nanoparticles, was retrieved and 100 ng, See FIG. 6, of material was added to 90 ul of cell culture medium overlaying approximately 50,000 adherent fibroblast cells (80% confluency, >95% viability). Cells were incubated for 24 h at 37° C.). During this time the cells endocytosed the mRNA-LNP's and produced the luciferase as coded for by the mRNA. The mRNA thus expresses a secreted luciferase, which is measured by obtaining supernatant and mixing that with substrate-containing buffer. Luciferase generated light is captured and used as quantitative measure for production of intact protein. A sample of LNPs from the same formulation batch, stored at 4° C., but not loaded into amylomatrix was used as positive control. Prior to release, the amylomatrix were stored dry for several weeks at 4° C. in closed vials.

It was measured that the amylomatrix shows about 10% expression [with no variation in between the samples] when compared to the positive control, consisting of LNP's not formulated in amylomatrix. The conclusion is that the amylomatrix is able to stabilize partly the mRNA within the LNP's, and that the processes of absorption, freezing, vacuum drying, storing and reconstitution can be used for stabilizing complete mRNA-LNP's, leaving them intact as to enable endocytosis and expression of luciferase.

### Discussion on Stability Factors

From experiments 5, 6 and 7, which all include the artificial biological constructs which are mRNA-LNP's, it can be concluded that the amylomatrix, when loaded with mRNA-LNP's, at least partly protects the mRNA from being chemically and/or enzymatically hydrolysed and/or from melting of its secondary structure; the amylomatrix simultaneously prevents at least partly the fusion and/or aggregation of the LNP's, the desintegration of the PEG-ylated lipids, the hexagonal transformation of the cationic ionizable lipids, the oxidation of lipids, the formation of membrane domains and/or the damage by physical stress factors; the result is that the amylomatrix at least stabilizes a part of the mRNA-LNP' in all those aspects in the same time, allowing endocytosis of the LNP's by the cell membrane, release of the mRNA from the endosomes into the cytosol and translation of the mRNA by the ribosomes into the coded for protein, which effectively showed its biological activity.

### Experiment 8: Stabilizing Bovine Herpes Virus

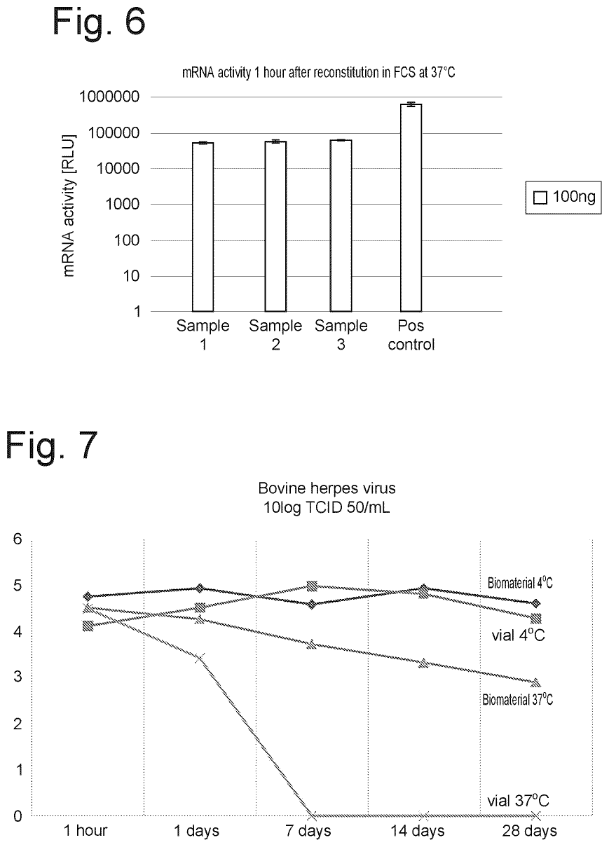

A number of vaccines consist of “living” viruses; these living viruses are either attenuated viruses [pathogenic viruses made non-pathogenic] or living viral vectors [mostly adenoviruses], that have been genetically modified by the introduction of a genetic code coding for proteins with epitopes of the pathogenic virus. In this experiment Bovine Herpes Virus I is used as an experimental virus, see results in FIG. 7.

### Preparation of the Samples.

Bovine Herpes Virus [BHV1] field strain Lam was grown as a test organism on embryonic bovine trachea cells. Plates were incubated for 5 days at 37° C. in air with 5% CO2. Virus titer of inoculum for the amylomatrix was 105.93 TCID50.

Hollow cylindrical rods 25 mm long and 2 mm diameter [inner diameter 1.2 mm] manufactured of amylomatrix were loaded with 30 μl viral suspension and the amylomatrix was allowed to absorb the viral suspension during 3 minutes. Subsequently the amylomatrix were frozen to −70° C. by bringing the vials in close contact with a metal block which was already at −70 C, as to create a snap freeze effect within 2 seconds. As a positive control the virus inocula, the same as used for the loading of the amylomatrix, was used and also frozen at −70° C. in a vial. The samples were stored at 4° C. or at 37° C. for 0 (1 hr), 1, 7, 14 or 28 days. For reconstitution, 1 ml water was added to the vials and the amylomatrix were dissolved.

### Results at 4° C.

BHV1 titer in the amylomatrix remained constant for the whole testperiod and BHV1 titer was as high as BHV1 titer in the reference samples. Mean BHV1 titer in the amylomatrix 1 hr after completion of the lyophilization process was 104.76 TCID50/ml. At the end of the testperiod, 28 days later, these titers were nearly the same, namely 104.61 TCID50/ml. No influence of storage temperature at 4 C was observed on BHV1 titer in reference samples. BHV1 titers after completion of the lyophilization process and at the end of the testperiod were for the reference of the samples 104.12 TCID50/ml and 104.29 TCID50/ml, respectively.

### Results at 37° C.

Storage conditions at 37° C. are better in amylomatrix than in the reference samples. BHV1 titer in reference samples were detected only until 1 day after completion of the lyophilization process whereas titers in amylomatrix were detected for the whole testperiod of 28 days. In the amylomatrix the titer declined from 104.52 TCID50 at 1 hour after completion of the lyophilization process till 102.90 TCID50, 28 days later. At day 3, the titer still was not much lower than the starting titer. It is concluded that Bovine Herpes Virus I is more stable at 37° C. when in amylomatrix than in the controls. As can be concluded from the graph, the biological construct BHV type 1 in amylomatrix can be stored and transported during 3 days at 37° C. without significant loss of viability, showing its biological activity. This result corresponds with the “3 days 40° C. challenge”, sufficient for the “last mile’ distribution of vaccines worldwide.

The present invention has been described above with reference to a number of exemplary embodiments as shown in the drawings. Modifications and alternative implementations of some parts or elements are possible, and are included in the scope of protection as defined in the appended claims.

1. A composition comprising (a) processed starch and (b) a virus is selected from one or more of the group consisting of Herpesviridae, Adenoviridae, Bunyaviridae, Filoviridae, Rhabdoviridae, Retroviridae, Reoviridae, Coronaviridae, Orthomyxoviridae, Paramyxoviridae, Togaviridae, Papillomaviridae, Poxviridae and Flaviviridae, wherein the processed starch comprises domains of at least partly intact amylopectin layers distributed throughout a matrix of homogeneous amylose phase.

2. The composition according to claim 1, wherein the virus is an attenuated virus, an inactivated virus, or a split virus.

3. The composition according to claim 1, wherein the virus is a genetically modified virus.

4. The composition according to claim 1, wherein the composition comprises a virus from the family of Togaviridae and a virus from the family of Paramyxoviridae.

5. The composition according to claim 1, wherein the composition has a water content of 0.5 to 5.0 wt. %.

6. The composition according to claim 1, wherein the composition comprises a stabilizing component selected from the group consisting of monosaccharides, disaccharides, trisaccharides, oligosaccharides, polysaccharides, glycoproteins, proteoglycans, peptidoglycans, glycolipids, lipopolysaccharides, and phosphonomannans.

7. The composition according to claim 1, wherein the composition comprises 5.0 to 99.9 wt. % of processed starch.

8. A process for storing biologically active constructs in a biodegradable material, the method comprising the steps:
a) providing a biodegradable material, wherein the biodegradable material comprises a processed starch, wherein the processed starch comprises domains of at least partly intact amylopectin layers distributed throughout a matrix of homogeneous amylose phase;
b) providing biologically active constructs;
c) absorbing said biologically active constructs into the biodegradable material; and
d) storing the biologically active constructs in the biodegradable material at a storage temperature from −80° C. to 80° C. for a period of at least 1 day,
wherein the biologically active constructs is a virus, a virus-like particle, or a virosome, wherein the virus is selected from one or more of the group consisting of Herpesviridae, Adenoviridae, Bunyaviridae, Filoviridae, Rhabdoviridae, Retroviridae, Reoviridae, Coronaviridae, Orthomyxoviridae, Paramyxoviridae, Togaviridae, Papillomaviridae, Poxviridae and Flaviviridae.

9. The process according to claim 8, wherein the process further comprises a step of cooling the biodegradable material comprising the biologically active constructs.

10. The process according to claim 9, wherein the cooling is performed within a period of 0.1 seconds to 30 seconds.

11. The process according to claim 9, wherein the cooling is commenced within 0.1 to 180 seconds following the start of the absorbing step.

12. The process according to claim 9, wherein the cooling is commenced within 45 to 600 seconds following the start of the absorbing step.

13. The process according to claim 8, wherein the process comprises, after the cooling step, a step of drying the biodegradable material comprising the biologically active constructs to a water content of less than 5 wt. %.

14. The process according to claim 8, wherein the process comprises a step of absorbing a stabilizing component into the biodegradable material comprising the biologically active construct, wherein the stabilizing component is selected from the group consisting of monosaccharides, disaccharides, trisaccharides, oligosaccharides, polysaccharides, glycoproteins, proteoglycans, peptidoglycans, glycolipids, lipopolysaccharides, and phosphonomannans.

15. The process according to claim 8, wherein the virus is an attenuated virus, an inactivated virus, or a split virus.

16. The composition according to claim 1, wherein
the composition has a water content of 1.0 to 4.0 wt. %
the composition comprises 10 to 99 wt. % of processed starch;
the composition comprises a stabilizing component that is a disaccharide; or
a combination thereof.

17. The composition according to claim 3, wherein the virus is a genetically modified virus from the family Adenoviridae.

18. The process according to claim 9, wherein
the cooling is performed within 10 seconds;
the cooling comprises cooling the biodegradable material to a temperature of −70° C. to −30° C.; or
a combination thereof.

19. The process according to claim 11, wherein
the cooling is commenced within 0.1 to 60 seconds following the start of the absorbing step;
the biodegradable material is provided as a rod-like, bullet-like, or needle-like shaped article having an inner, hollow portion and an average wall thickness of 10 to 2500 μm; or
a combination thereof.

20. The process according to claim 12, wherein
the cooling is commenced within 60 to 300 seconds following the start of the absorbing step;
the biodegradable material is provided as a tablet or a capsule; or
a combination thereof.
